SEQUENCE LISTING (4 sequences)

SEQ ID NO: 1 - protein, 9 aa, synthetic construct
  REGION 1..9: note = Synthetic
  VARIANT 5: note = Xaa can be any naturally occurring amino acid
GDVEXNPGP

SEQ ID NO: 2 - protein, 8 aa, synthetic construct
  REGION 1..8: note = Synthetic
GDVESNPG

SEQ ID NO: 3 - protein, 7 aa, synthetic construct
  REGION 1..7: note = Synthetic
DVEENPG

SEQ ID NO: 4 - protein, 9 aa, synthetic construct
  REGION 1..9: note = Synthetic
AGDVEENPG

(19) United States
(12) Patent Application Publication
(10) Pub. No.: US 2023/0136076 A1
(43) Pub. Date: May 4, 2023

(54) METHODS OF DETERMINING GENE EDITING EFICIENCIES IN CELLS

(71) Applicant: Pact Pharma, Inc., South San Francisco, CA (US)

(72) Inventors: Kyle JACOBY, Emeryville, CA (US); Barbara Sennino, San Francisco, CA (US); Susan Foy, South San Francisco, CA (US); Theresa L. Hunter, South San Francisco, CA (US)

(73) Assignee: Pact Pharma, Inc., South San Francisco, CA (US)

(21) Appl. No.: 18/049,114

(22) Filed: Oct. 24, 2022

Related U.S. Application Data
(63) Continuation of application No. PCT/US2021/028829, filed on Apr. 23, 2021.
(60) Provisional application No. 63/015,067, filed on Apr. 24, 2020.

(51) Int. Cl. G01N 15/14; G01N 33/569; C12N 15/90; C07K 7/08

(52) U.S. Cl. CPC G01N 15/1459; G01N 33/56966; C12N 15/907; C07K 7/08; G01N 1/30

(57) ABSTRACT

Methods of detecting the efficacy and presence of a biologic drug which uses one or more 2A peptide in its manufacture and/or final pharmaceutical formulation.

### CROSS REFERENCE TO RELATED APPLICATIONS

This Application is a Continuation of International Patent Application No. PCT/US21/28829, filed on Apr. 23, 2021, which claims priority to U.S. Provisional Application No. 63/015,067, filed on Apr. 24, 2020, the content of each of which is incorporated by reference in its entirety, and to each of which priority is claimed.

### SEQUENCE LISTING

The instant application contains a Sequence Listing which has been submitted electronically in XML format and is hereby incorporated by reference in its entirety. Said XML copy, created on Jan. 3, 2023, is named 087520.0270.xml and is 6,349 bytes in size.

### BACKGROUND OF THE INVENTION

2A peptides can be used to facilitate the co-express multiple genes. 2A peptides are particularly advantageous in this role because they can lead to high levels of downstream protein expression relative to other multi-gene co-expression strategies. Additionally, the small size of the 2A peptide is beneficial because it results in a lower risk of interference with the function of genes being co-expressed. Despite the benefits associated with the use of 2A peptides as a mechanism for co-expression of multiple genes, or multiple variations of the same gene, one drawback of conventional 2A-based strategies is the inability to confirm insertion of the co-expressed genes into the genome of the targeted cell. This is especially apparent when distinct sets of genes are inserted into an array of cells. This problem is compounded when the insertion of the new genes into the genome of a cell is accomplished in such a way that the 2A peptide(s) are cleaved during translation such that some of the proteins expressed from the inserted genes are no longer linked to the 2A peptides. Indeed, it is possible, and in some instances advantageous, to engineer the insertion of the co-expressed genes to include additional cleavage sites sufficient to remove all 2A fragments from the translated proteins of interest. This is a desirable approach, despite its impact on the ability to confirm insertion of the co-expressed genes into a target cell, because it allows the inserted protein to be “native” and not include 2A fragments that could lead to non-endogenous epitopes. Accordingly, there is a need to find efficient and reliable screening methods to detect the successful insertion of genes into the genome of a target cell that makes use of the presence of self-cleaving 2A peptides, but which are not dependent on the 2A peptide, or a fragment thereof, remaining attached to the translated protein(s) of interest.

### SUMMARY OF THE INVENTION

The present disclosure provides a method of determining a presence of an adoptive cell therapy in a subject, comprising: detecting an expression level of a 2A peptide in a sample from the subject that has received the adoptive cell therapy, wherein the adoptive cell therapy comprises a cell expressing the 2A peptide, or a fragment thereof.

In certain embodiments, detection of the 2A peptide, or a fragment thereof, is associated with the presence of the adoptive cell therapy. In certain embodiments, absence of the 2A peptide, or a fragment thereof, is associated with inefficiency of the adoptive cell therapy. In certain embodiments, detecting of the 2A peptide, or a fragment thereof, occurs between 1 day and 30 days after the subject has received the adoptive cell therapy. In certain embodiments, detecting of the 2A peptide, or fragment thereof, occurs between 1 month and 12 months after the subject has received the adoptive cell therapy. In certain embodiments, detecting of the 2A peptide or fragment thereof occurs between 1 year and 5 years after the subject has received the adoptive cell therapy.

Further, the present disclosure provides a method of monitoring the persistence of an adoptive cell therapy in a subject that has received the adoptive cell therapy, comprising: detecting an expression level of a 2A peptide, or fragment thereof, in a sample from a first time point from the subject; and detecting the expression level of the 2A peptide, or fragment thereof, in a sample from a second time point from the subject; wherein the adoptive cell therapy comprises a cell expressing the 2A peptide, and wherein detection of the 2A peptide, or fragment thereof, in the sample of the second time point is associated with the persistence of the adoptive cell therapy. The present disclosure also provides a method of monitoring the persistence of an adoptive cell therapy in a subject that has received the adoptive cell therapy, comprising: detecting an expression level of a 2A peptide, or fragment thereof, in a sample from a first time point from the subject; and detecting the expression level of the 2A peptide, or fragment thereof, in a sample from a second time point from the subject; wherein the adoptive cell therapy comprises a cell expressing the 2A peptide, and wherein absence of the 2A peptide, or fragment thereof, in the sample of the second time point is associated with the inefficiency of the adoptive cell therapy. In certain embodiments, the method further comprises administering a second adoptive cell therapy to the subject.

The present disclosure provides a method of monitoring the expansion of an adoptive cell therapy in a subject that has received the adoptive cell therapy, comprising: detecting an amount of cells expressing a 2A peptide, or fragment thereof, in a sample from a first time point from the subject; and detecting an amount of cells expressing a 2A peptide, or fragment thereof, in a sample from a second time point from the subject; wherein expansion of the adoptive cell therapy occurs when the amount of cells expressing a 2A peptide, or fragment thereof, in a sample of the second time point is increased compared to the amount of cells expressing a 2A peptide or fragment thereof in a sample of the first time point. The present disclosure also provides a method of monitoring the expansion of an adoptive cell therapy in a subject that has received the adoptive cell therapy, comprising: detecting an amount of cells expressing a 2A peptide or fragment thereof in a sample from a first time point from the subject; and detecting an amount of cells expressing a 2A peptide or fragment thereof in a sample from a second time point from the subject; wherein no expansion of the adoptive cell therapy occurs when the amount of cells expressing a 2A peptide or fragment thereof in a sample of the second time point is decreased or the same compared to the amount of cells expressing a 2A peptide or fragment thereof in a sample of the first time point. In certain embodiments, the second time point occurs between 1 day and 30 days after the first time point. In certain embodiments, the second time point occurs between 1 month and 12 months after the first time point. In certain embodiments, the second time point occurs between 1 year and 5 years after the first time point.

In certain embodiments, the cell is a Cell Product. In certain embodiments, the cell is a NeoTCR Product. In certain embodiments, the sample is a blood sample. In certain embodiments, the sample is a tumor sample. In certain embodiments, the subject is human. In certain embodiments, the subject has a cancer.

In certain embodiments, the 2A peptide, or fragment thereof, is detected by flow cytometry. In certain embodiments, the 2A peptide, or fragment thereof, is detected by immunohistochemistry (IHC). In certain embodiments, the IHC is ChipCytometry.

The present disclosure further provides a method for the prognosis of a subject treated with an adoptive cell therapy, comprising: detecting the expression level of a 2A peptide, or fragment thereof, in a sample from the subject wherein the adoptive cell therapy comprises a cell expressing the 2A peptide, or fragment thereof, and determining the expression level of the 2A peptide, or fragment thereof, in a sample from the subject, wherein detection of the 2A peptide, or fragment thereof, is associated with efficacy of the adoptive cell therapy. The present disclosure also provides a method for the prognosis of a subject treated with an adoptive cell therapy, comprising: detecting the expression level of a 2A peptide, or fragment thereof, in a sample from the subject wherein the adoptive cell therapy comprises a cell expressing the 2A peptide, or fragment thereof, and determining an expression level of a 2A peptide, or fragment thereof, in a sample from the subject, and wherein no detection of the 2A peptide, or fragment thereof, is associated with reduced efficacy of the adoptive cell therapy. In certain embodiments, the method further comprises administering a second adoptive cell therapy to the subject.

In certain embodiments, detecting of the 2A peptide, or fragment thereof, occurs between 1 day and 30 days after the subject has received the adoptive cell therapy. In certain embodiments, detecting of the 2A, or fragment thereof, peptide occurs between 1 month and 12 months after the subject has received the adoptive cell therapy. In certain embodiments, detecting of the 2A, or fragment thereof, peptide occurs between 1 year and 5 years after the subject has received the adoptive cell therapy.

In certain embodiments, the cell is a Cell Product. In certain embodiments, the cell is a NeoTCR Product. In certain embodiments, the sample is a blood sample. In certain embodiments, the sample is a tumor sample. In certain embodiments, the subject is human. In certain embodiments, the subject has a cancer.

In certain embodiments, the 2A peptide, or fragment thereof, is detected by flow cytometry. In certain embodiments, the 2A peptide, or fragment thereof, is detected by IHC. In certain embodiments, the IHC is ChipCytometry.

The present disclosure also provides a method of treating a cancer in a subject that has received an adoptive cell therapy, wherein the adoptive cell therapy comprises a cell expressing a 2A peptide, or fragment thereof, the method comprising: detecting the expression level of the 2A peptide, or fragment thereof, in a sample from the subject, wherein absence or decreased detection of the 2A peptide, or fragment thereof, is associated with inefficiency of the adoptive cell therapy; and administering a second adoptive cell therapy, when the expression level of the 2A peptide is absent or decreased.

In certain embodiments, detecting of the 2A peptide, or fragment thereof, occurs between 1 day and 30 days after the subject has received the adoptive cell therapy. In certain embodiments, detecting of the 2A peptide, or fragment thereof, occurs between 1 month and 12 months after the subject has received the adoptive cell therapy. In certain embodiments, detecting of the 2A peptide, or fragment thereof, occurs between 1 year and 5 years after the subject has received the adoptive cell therapy.

In certain embodiments, the cell is a Cell Product. In certain embodiments, the cell is a NeoTCR Product. In certain embodiments, the sample is a blood sample. In certain embodiments, the sample is a tumor sample. In certain embodiments, the subject is human.

In certain embodiments, the 2A peptide, or fragment thereof, is detected by flow cytometry. In certain embodiments, the 2A peptide or fragment thereof is detected by IHC. In certain embodiments, the IHC is ChipCytometry.

In certain embodiments, the 2A peptide, or fragment thereof, is a cleaved 2A peptide. In certain embodiments, the 2A peptide is selected from the group comprising P2A, T2A, E2A, and F2A peptides. In certain embodiments, the 2A peptide is a P2A or T2A peptide. In certain embodiments, the 2A peptide is a P2A peptide.

In certain embodiments, the 2A peptide, or fragment thereof, is detected using an anti-2A antibody or antigen binding portion thereof. In certain embodiments, the anti-2A antibody cross-competes for binding to the 2A peptide with a reference antibody or antigen-binding portion thereof, wherein the reference antibody or antigen-binding portion is a 3H4 antibody. In certain embodiments, the anti-2A antibody binds to the same epitope of the 2A peptide with a reference antibody or antigen-binding portion thereof, wherein the reference antibody or antigen-binding portion thereof, wherein the reference antibody or antigen-binding portion is a 3H4 antibody. In certain embodiments, the anti-2A antibody cross-competes for binding to the 2A peptide with a reference antibody or antigen-binding portion thereof, wherein the reference antibody or antigen-binding portion is an ABS31 antibody. In certain embodiments, the anti-2A antibody binds to the same epitope of the 2A peptide with a reference antibody or antigen-binding portion thereof, wherein the reference antibody or antigen-binding portion thereof, wherein the reference antibody or antigen-binding portion is an ABS31 antibody. In certain embodiments, the anti-2A antibody or antigen-binding portion thereof is a 3H4 antibody. In certain embodiments, the anti-2A antibody or antigen-binding portion thereof is an ABS31 antibody. In certain embodiments, the anti-2A antibody binds to an epitope having an amino acid sequence set forth in SEQ ID NO: 3.

The present disclosure provides a method of determining whether a cell has been edited, comprising transducing or transfecting a cell with a vector encoding an antigen binding receptor and a 2A peptide; detecting an expression level of the 2A peptide, or fragment thereof, in the cell; wherein detection of the 2A peptide, or fragment thereof, is associated with efficiency of gene editing. The present disclosure also provides a method of determining whether a cell has been edited, comprising transducing or transfecting a cell with a vector encoding an antigen binding receptor and a 2A peptide; detecting an expression level of the 2A peptide, or fragment thereof, in the cell; wherein absence of the 2A peptide, or fragment thereof, is associated with inefficiency of gene editing. The present disclosure provides the antigen binding receptor is a T cell receptor (TCR).

The present disclosure provides the vector comprises first and second homology arms homologous to first and second target nucleic acid sequences and oriented to facilitate homologous recombination; a nucleotide sequence encoding the TCR positioned between the first and second homology arms; and a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide, wherein the first and second nucleotide sequences encoding the 2A ribosome skipping elements have the same amino acid sequences and are codon-diverged relative to each other.

The present disclosure further provides a method of lot releasing a population of cells for adoptive cell therapy, comprising: transducing or transfecting the population of cells with a vector comprising an antigen binding receptor and a 2A peptide; detecting an amount of cells expressing the 2A peptide, or fragment thereof; releasing the population of cells for therapeutic use when the amount of cells expressing the 2A peptide is above a threshold. In certain embodiments, the antigen binding receptor is a T cell receptor (TCR). In certain embodiments, the vector comprises first and second homology arms homologous to first and second target nucleic acid sequences and oriented to facilitate homologous recombination; a nucleotide sequence encoding the TCR positioned between the first and second homology arms; and a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide, wherein the first and second nucleotide sequences encoding the 2A ribosome skipping elements have the same amino acid sequences and are codon-diverged relative to each other. In certain embodiments, the threshold is calculated by the formula:

$$\frac{amount of cells expressing the 2 A peptide}{amount of cells in the population of cell} \times 100 \% .$$

In certain embodiments, the threshold is equal to or greater than 5%. In certain embodiments, the population of cell comprises a Cell Product. In certain embodiments, the population of cell comprises a NeoTCR Product.

In certain embodiments, the 2A peptide, or fragment thereof, is detected by flow cytometry. In certain embodiments, the 2A peptide, or fragment thereof, is detected by IHC. In certain embodiments, the IHC is ChipCytometry.

In certain embodiments, the 2A peptide is a cleaved 2A peptide. In certain embodiments, the 2A peptide is selected from the group comprising P2A, T2A, E2A, and F2A peptides. In certain embodiments, the 2A peptide is P2A or T2A. In certain embodiments, the 2A peptide is P2A.

In certain embodiments, the 2A peptide, or fragment thereof, is detected using an anti-2A antibody or antigen binding portion thereof. In certain embodiments, the anti-2A antibody cross-competes for binding to the 2A peptide with a reference antibody or antigen-binding portion thereof, wherein the reference antibody or antigen-binding portion is a 3H4 antibody. In certain embodiments, the anti-2A antibody binds to the same epitope of the 2A peptide with a reference antibody or antigen-binding portion thereof, wherein the reference antibody or antigen-binding portion thereof, wherein the reference antibody or antigen-binding portion is a 3H4 antibody. In certain embodiments, the anti-2A antibody cross-competes for binding to the 2A peptide with a reference antibody or antigen-binding portion thereof, wherein the reference antibody or antigen-binding portion is an ABS31 antibody. In certain embodiments, the anti-2A antibody binds to the same epitope of the 2A peptide with a reference antibody or antigen-binding portion thereof, wherein the reference antibody or antigen-binding portion thereof, wherein the reference antibody or antigen-binding portion is an ABS31 antibody. In certain embodiments, the anti-2A antibody or antigen-binding portion thereof is a 3H4 antibody. In certain embodiments, the anti-2A antibody or antigen-binding portion thereof is an ABS31 antibody. In certain embodiments, the anti-2A antibody binds to an epitope having an amino acid sequence set forth in SEQ ID NO: 3.

The present disclosure provides a method of treating a patient with a Cell Product, the method comprising: administering a patient in need thereof a Cell Product, drawing blood or collecting a tumor sample from the patient at a future time point, and analyzing the blood or tumor sample using FACS or IHC analysis to determine if the 2A peptide or antibody binding fragment thereof is present using an anti-2A antibody, wherein if the 2A peptide or antibody binding fragment thereof is detected then the Cell Product is present and persisting in the patient and if the 2A peptide or antibody binding fragment thereof is not detected then the Cell Product is no longer in the patient, and wherein if the Cell Product is present and persisting, the product is effective at implanting into the patient and no additional therapy is administered to the patient, and wherein if the Cell Product is not present, the product is not effective at implanting into the patient and a new therapy is administered to the patient. In certain embodiments, the Cell Product is a NeoTCR product. In certain embodiments, the patient is a patient with cancer. In certain embodiments, the future time point is selected from a time point between 1-10 days, 11-30 days, 31-90 days, 91-180 days, and 181-365 days. In certain embodiments, the future time point is selected from a time point between 1-2 years, 2-5 years, 5-10 years, 10-20 years, and 20-50 years.

The present disclosure provides a method of detecting a cleaved 2A peptide or antibody binding fragment thereof in cells of a Cell Product using FACS, comprising one of the following methods: a) Method 1, comprising: staining the cells with a live/dead label, fixing the cells with a fixative, staining the cells with cell surface markers of interest, staining the cells with a permeabilization buffer with an anti-2A antibody, fixing the cells with a fixative; b) Method 2, comprising: staining the cells with a live/dead label, staining the cells with cell surface markers of interest, fixing the cells with a fixative, staining the cells with a permeabilization buffer with an anti-2A antibody, fixing the cells with a fixative; and c) Method 3 comprising: staining the cells with a live/dead label, staining the cells with cell surface markers of interest, staining the cells with a permeabilization buffer with an anti-2A antibody, fixing the cells with a fixative; wherein the stained cells from Methods 1, 2, or 3 are analyzed on a flow cytometer using FACS analysis. In certain embodiments, the cells are gene edited using the methods described in Example 2 and/or 3.

The present disclosure provides a method of determining the prognosis of a patient treated with a Cell Product, the method comprising: administering a patient in need thereof a Cell Product, drawing blood or collecting a tumor sample from the patient at a future time point, and analyzing the blood or tumor sample using FACS or IHC analysis to determine if the 2A peptide or antibody binding fragment thereof is present using an anti-2A antibody, wherein if the 2A peptide or antibody binding fragment thereof is detected then the Cell Product is present and persisting in the patient and if the 2A peptide or antibody binding fragment thereof is not detected then the Cell Product is no longer in the patient, and wherein if the Cell Product is present and persisting, the prognosis of the patient is good for responding to the Cell Product therapy, and wherein if the Cell Product is not present, the prognosis of the patient is bad for responding to the Cell Product therapy. In certain embodiments, the Cell Product is a NeoTCR product. In certain embodiments, the patient is a patient with cancer. In certain embodiments, a good prognosis is partial or complete response to the Cell Therapy. In certain embodiments, a bad prognosis is no response to the Cell Therapy and/or disease progression.

The present disclosure provides a method of determining the persistence and/or presence of a Cell Product in a patient after infusion of the Cell Product, the method comprising: drawing blood or collecting a tumor sample from the patient at a time point following administration of the Cell Product, and analyzing the blood or tumor sample using FACS or IHC analysis to determine if the 2A peptide or antibody binding fragment thereof is present using an anti-2A antibody, wherein the Cell Product is present and has persisted in the patient if the 2A peptide or antibody binding fragment thereof is detected using the anti-2A antibody. The present disclosure provides a method of determining the expansion of a Cell Product in a patient after infusion of the Cell Product, the method comprising: drawing blood or collecting a tumor sample from the patient at two or more time points following administration of the Cell Product, analyzing the blood or tumor samples using FACS or IHC analysis to determine if the 2A peptide or antibody binding fragment thereof is present using an anti-2A antibody, and determining the difference between the number of cells stained with the anti-2A antibody at the two or more timepoints, wherein, an increase in the cell numbers stained with the anti-2A antibody between a first time point and a second is evidence of cell expansion, and wherein a decrease or no increase in the cell numbers stained with the anti-2A antibody between a first time point and a second is evidence of no cell expansion.

The present disclosure provides a method for detecting the rate of gene editing of a gene editing reaction, the method comprising: permeabilizing and staining the cells from the gene editing reaction with an anti-2A peptide antibody, sorting the cells using FACS or IHC to identify the number of unstained and stained cells, and calculating the rate of gene editing using the following calculation: (number of cells positive for anti-2A antibody staining)□(total number of cells). In certain embodiments, the permeabilizing and staining the cells is selected from the group consisting of Method 1, Method 2, and Method 3 disclosed herein.

The present disclosure provides a method for qualifying a Cell Product for release for the treatment of patients in need thereof, the method comprising: taking a sample of the Cell Product, analyzing the sample using FACS or IHC analysis to determine if the 2A peptide or antibody binding fragment thereof is present using an anti-2A antibody, and determining the percent of edited cells, wherein, the Cell Product is released for the treatment of patients if the percent of edited cells meets the product specification for release. In certain embodiments, the percent of edited cells is determined using the following formula: (number of cells positive for anti-2A antibody staining)/(total number of cells). In certain embodiments, the product specification for release is at least 5% gene edited cells.

In certain embodiments, the future time point is selected from a time point between 1-10 days, 11-30 days, 31-90 days, 91-180 days, and 181-365 days. In certain embodiments, the future time point is selected from a time point between 1-2 years, 2-5 years, 5-10 years, 10-20 years, and 20-50 years. In certain embodiments, the anti-2A antibody binds to the epitope DVEENPG (SEQ IN NO: 3). In certain embodiments, the anti-2A antibody is the 3H4 or the ABS31 antibody. In certain embodiments, the anti-2A antibody competes for binding with the 3H4 and/or ABS31 antibody. In certain embodiments, the anti-2A antibody binds the same epitope as the 3H4 and/or ABS31 antibody. In certain embodiments, the IHC is ChipCytometry.

### BRIEF DESCRIPTION OF THE DRAWINGS

FIG. 1. FIG. 1 shows the sequences of the 2A peptides. The conserved C-terminal sequence of the 2A peptides is shown in bold for each of the peptides. The single underlined sequence of the T2A peptide represents the epitope allegedly recognized by the Novus Biologicals 3H4 monoclonal antibody designed for Western Blot detection of T2A. The double underlined sequence of the P2A peptide represents the P2A epitope used for detection by FACS and disclosed herein.

FIGS. 2A and 2B. FIG. 2A shows a diagram of a plasmid template wherein a gene of interest is flanked by 2A peptides. As shown, the gene of interest is also flanked by signal sequences to enable targeting and trafficking of the expressed gene of interest, an optional protease cleavage site that can be used to remove non-native amino acids from the gene product of interest that could otherwise result from having amino acids from the 2A peptide remaining attached to the gene of interest, and the 2A peptides. FIG. 2B shows the translated sequence (top) and the post-translational processing (bottom). integration of the gene of interest into the desired locus and the translation of such gene. The constructs shown in FIGS. 2A and 2B also include a codon optimized sequence. The codon optimized sequence was included to restore functional expression of the endogenous target gene while inserting an additional gene under the native control of that gene's promoter/regulatory elements. Alternatively, while not shown, a codon optimized sequence is not needed in order for the 2A Diagnostic described herein to work. For example, it is possible to simply disrupt the target gene, and remove its expression but still use the 2A Diagnostic to see expression of the inserted gene of interest.

FIGS. 3A and 3B. FIGS. 3A and 3B show the neoantigen-specific TCR construct design used for integrating neoantigen-specific TCR constructs (neoTCRs) into the TCRα locus. FIG. 3A shows the target TCRα locus (endogenous TRAC, top panel) and its CRISPR Cas9 target site (horizontal stripe, cleavage site designated by arrow), and the circular plasmid HR template (bottom panel) with the polynucleotide encoding the neoTCR, which is located between left and right homology arms (“LHA” and “RHA” respectively) prior to integration. FIG. 3B shows the integrated neoTCR in the TCRα locus (top panel), the transcribed and spliced neoTCR mRNA (middle panel), and translation and processing of the expressed neoTCR (bottom panel).

FIGS. 4A and 4B. FIG. 4A provides a high-level schematic diagraming the detection and staining of the cleaved 2A peptide in gene edited cells. FIG. 4B provides a high-level diagram of the detection and staining of the cleaved 2A peptide in T cells that were gene edited to express a neoTCR. As shown in both FIG. 4A and FIG. 4B, the 2A peptide is cleaved off of the gene of interest to avoid the creation of a non-endogenous epitope on the gene of interest which could otherwise cause an immunogenic response in a patient. The cleaved 2A peptides are shown to be located in the endoplasmic reticulum and detectable using FACS after permeabilizing the cells (as shown with the saponin permeabilization and antibody staining in the righthand portion of the figures).

FIGS. 5A and 5B. FIG. 5A shows two FACS plots gated on live cells using an anti-2A antibody conjugated to AlexaFluor 467 in permeabilized cells. The upper plot shows the anti-2A antibody (i.e., AlexaFluor 467) signal in the NeoTCR-2 transfected cells but not in the mock transfected (control) cells. FIG. 5B shows two FACS plots gated on cells that were positive for lymphocytes, singlets, live cells, and CD8+. The light colored plot is under permeabilization conditions and the dark colored plot is under non-permeabilization conditions. As shown in the mock transfection (control cells) FACS plot on the left, there is no identifiable 2A peptide signal in permeabilized or non-permeabilized when there is no editing with 2A peptides. As shown in the right hand FACS plot, cells that were edited using 2A peptides exhibit a detectable anti-2A peptide signal when the cells are permeabilized but not when they are not permeabilized.

FIG. 6. FIG. 6 shows three columns of FACS plots: left hand column (Control; Fluorescence Minus One Control (FMO)); middle column (Control; Isotype, cells stained with APC un-conjugated to an anti-2A antibody); right column (cells stained with an anti-2A antibody conjugated to AlexaFluor 467). All experiments were performed in permeabilized cells. As shown in the upper right quadrants of the anti-2A×NeoTCR-2 and anti-2A×NeoTCR-1 plots, 2A peptide was detected in gene edited cells (editing done using 2A peptides in the construct). The control and mock transfected plots show no 2A staining. This shows that expression of the neoTCR on the cell surface is confirmed with a neoTCR-specific dextramer (i.e., there is co-staining of the 2A peptide or cleaved portion thereof and the neoTCR-specific dextramer).

FIG. 7. FIG. 7 shows dextramer (staining for a specific NeoTCR) and 2A peptide staining on CD4 T cells gated for lymphocytes positive for singlets, live cells, and CD4+.

FIG. 8. FIG. 8 shows FACS plots showing dextramer (NeoTCR) and 2A peptide staining on unstimulated CD8+ T cells or CD8+ T cells stimulated overnight for 18 hours with TransAct. The gating for this experiment was positive for lymphocytes, singlets, live cells, and CD8+ cells.

FIG. 9. FIG. 9 shows FACS plots showing dextramer (NeoTCR) and 2A peptide staining on gene edited T cells stimulated for 24 hours by co-culture with cells pulsed with 1000 nM, 100 nM, 10 nM, 1 nM, 0.1 nM, 0.01 nM, and 0 nM.

FIG. 10. FIG. 10 shows data from cells that were fixed under specific conditions for 5 min before proceeding with normal staining procedures. Edited cells were assessed using dextramer (neoTCR) or 2A peptide staining. The conditions were (in order from left to right in the graph): 1) intracellular (IC) fixation buffer after dextramer staining, 2) intracellular (IC) fixation buffer after surface protein staining, 3) 1.6% paraformaldehyde (PFA) fixation after dextramer staining, 4) 1.6% paraformaldehyde (PFA) fixation after surface protein staining, and 5) no fixation.

FIG. 11. FIG. 11 shows the separation index of 2A staining after various fixation methods. The conditions were (in order from left to right in the graph): 1) 4% paraformaldehyde (PFA) fixation, 5 min, 2) 4% paraformaldehyde (PFA) fixation, 10 min, 3) intracellular (IC) fixation buffer, 5 min, 4) intracellular (IC) fixation buffer, 10 min, 5) fixation and permeabilization, 6) 1.6% paraformaldehyde (PFA) fixation, 5 min, 7) 1.6% paraformaldehyde (PFA) fixation, 10 min, and 8) no fixation.

FIGS. 12A and 12B. FIG. 12A shows FACS plots showing dextramer (NeoTCR) and TCRαβ staining on unstimulated CD8+ T cells or CD8+ T cells stimulated for one two days with TransAct. FIG. 12B shows FACS plots showing dextramer (NeoTCR) and 2A peptide staining on unstimulated CD8+ T cells or CD8+ T cells stimulated for one two days with TransAct.

FIGS. 13A and 13B. FIG. 13A shows the 2A+ cell frequency detected in blood samples from patients who were administered a NeoTCR Product. FIG. 13B shows the number of 2A+ cells detected in blood samples from patients who were administered a NeoTCR Product.

FIG. 14. FIG. 14 shows a serial dilution assay to determine the correct dilution of the 3H4 anti-2A antibody for ChipCytometry analysis.

FIG. 15A and 15B. FIG. 15A shows the IHC staining of gene edited cells using the 3H4 anti-2A antibody. FIG. 15B is a magnified view of a cell from FIG. 15A.

FIG. 16. FIG. 16 shows two sectioned images of the same cell using IHC, specifically ChipCytometry. As shown, the anti-2A antibody can detect intracellular 2A peptide (and cleaved fragments of the 2A peptide as described herein).

### DETAILED DESCRIPTION OF THE INVENTION

The present disclosure is based, in part, on the surprising result that 2A peptides can be detected in genetically modified cells, e.g. a NeoTCR Cell, targeting cell expressing antigens. The present disclosure thus provides methods for diagnosis and prognosis of a patient's disease susceptible to treatment with adoptive cell therapies as well as determining the efficiency of such adoptive cell therapies, e.g., the presence, persistence, and/or expansion of adoptive call therapies. In addition, the present disclosure provides methods for determining efficiency of gene editing of cells used in adoptive cell therapies. The present disclosure also provides methods for the lot release of a population of cells for adoptive cell therapy based on determining the efficiency of gene editing in the population of cells.

Non-limiting embodiments of the present disclosure are described by the present description and examples. For purposes of clarity of disclosure and not by way of limitation, the detailed description is divided into the following subsections:
1. Definitions;
2. Adoptive Cell Therapies;
3. 2A Peptides;
4. Methods of Use;
5. Kits; and
6. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the invention described herein include “comprising”, “consisting”, and “consisting essentially of” aspects and embodiments. The terms “comprises” and “comprising” are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, the term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antigen binding portion thereof” refers to a molecule other than an intact antibody that comprises a portion of an intact antibody that binds the antigen to which the intact antibody binds. Examples of antigen binding portion include, but are not limited to, Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments.

The term “2A Diagnostic” refers to a method of detecting a 2A peptide, or an antibody binding fragment thereof, or an antibody, or antigen binding portion thereof, in a patient who was administered an adoptive cell therapy, e.g., a Cell Product, or in the adoptive cell therapy itself. In certain embodiments, methods of detecting a 2A peptide, or fragment thereof, described herein are referred to as “2A Diagnostics.” In certain embodiments, the 2A peptide, or an antibody binding fragment thereof, or the antibody, or antigen binding portion thereof can be detected using 2A Diagnostics. In certain embodiments, the 2A Diagnostics can use an anti-2A peptide antibody. In certain embodiments, any antibody, or antigen binding portion thereof, that binds the translated 2A peptide can be used. In certain embodiments, the anti-2A antibody, or antigen binding portion thereof, is conjugated to a detection moiety. In certain embodiments, the detection moiety is a fluorophore.

“2A” and “2A peptide” are used interchangeably herein and mean a class of 18-22 amino acid long, viral, self-cleaving peptides that are able to mediate cleavage of peptides during translation in eukaryotic cells.

As used herein, the terms “Cancer” and “Tumor” are used interchangeably and refer to all neoplastic cell growth and proliferation, whether malignant or benign, and all pre-cancerous and cancerous cells and tissues. The terms are further used to refer to or describe the pathophysiological condition in mammals that is typically characterized by unregulated cell growth/proliferation. Examples of cancer include, but are not limited to, those described herein. Cancer can affect a variety of cell types, tissues, or organs, including, but not limited to, an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). The terms “Cancer” or “Tumor” and “Proliferative Disorder” are not mutually exclusive as used herein.

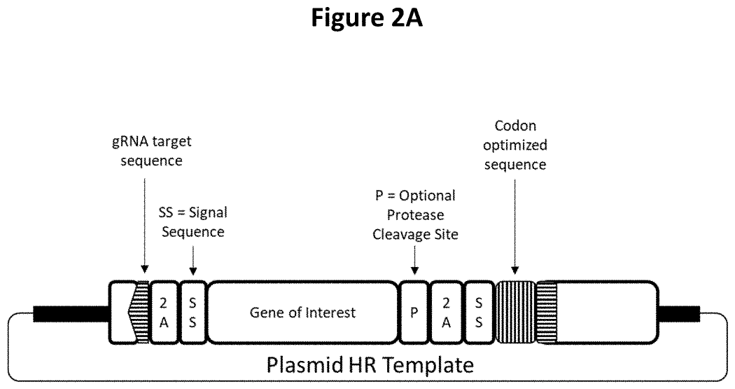

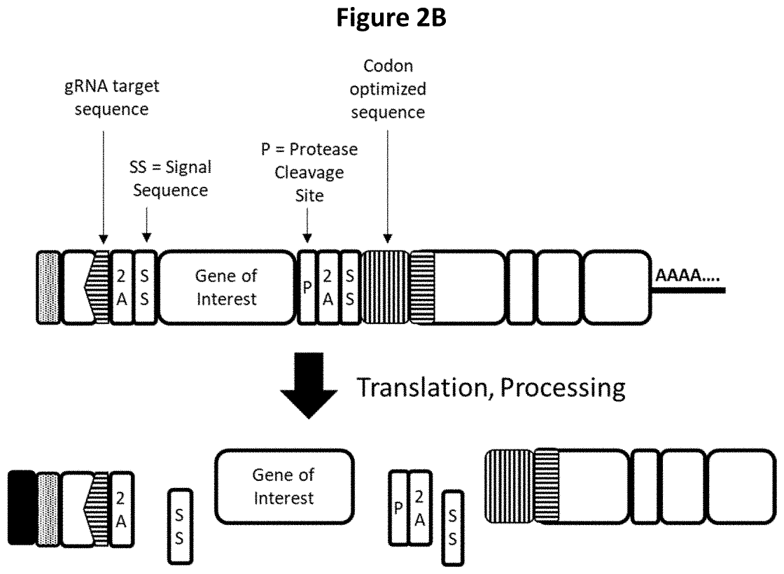

The term “Cell Product”, as used herein, means a gene edited cell therapy wherein one or more 2A peptides are used in the gene editing process. In certain embodiments, the Cell Product is made through the insertion of DNA wherein the gene of interest is inserted between two 2A sequences (see, e.g., FIG. 2A). In certain embodiments, the DNA is linear or circular (e.g., plasmid DNA). In certain embodiments, the Cell Product is made through the insertion of DNA wherein the gene of interest is flanked on one side by a 2A peptide. In certain embodiments, when there are more than one 2A peptide sequence, such sequences are the same 2A peptides (e.g., two P2A sequences, two T2A sequences, two E2A sequences, or 2 F2A sequences). In certain embodiments, when there are more than one 2A peptide sequence, such sequences are different 2A peptides (e.g., but not limited to, one T2A and one P2A). In certain embodiments, Cell Products are made using viral gene editing methods. In certain embodiments, Cell Products are made using targeted or non-targeted viral methods. In certain embodiments, Cell Products are made using non-viral gene editing methods, e.g., electroporation. Cell Products include, but without any limitation, T cell, NK cell, HSCs, TILs, and cells derived from HSCs. In certain embodiments, Cell Products can also include any other naturally occurring cell that can be edited using a 2A peptide as part of the gene editing process. Cell Products can be used, for example, for the treatment of autoimmune diseases, neurological diseases and injuries (including but not limited to Alzheimer's disease, Parkinson's disease, spinal cord and nerve injuries and/or damage), cancer, infectious diseases, joint disease (including but not limited to rebuilding damaged cartilage in joints), improving the immune system, cardiovascular disease and abnormalities, aging, immune deficiencies (including but not limited to multiple sclerosis and amyotrophic lateral sclerosis), allergies, and genetic disorders. In certain embodiments, Cell Products include NeoTCR Products and NeoTCR Viral Products.

“NeoTCR” and “neoTCR” are used interchangeably herein and means a neoepitope-specific T cell receptor that is introduced into a T cell, e.g., by gene editing methods.

As used herein, the term “NeoTCR cells” means one or more cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product is administered.

As used herein, the term “NeoTCR Product” refers to a pharmaceutical formulation comprising one or more NeoTCR cells. A NeoTCR Product comprises autologous precision genome-engineered CD8+ and CD4+ T cells. Using a targeted DNA-mediated non-viral precision genome engineering approach, expression of the endogenous TCR is disrupted and replaced by a patient-specific NeoTCR. In certain embodiment, the NeoTCR is isolated from peripheral CD8+ T cells targeting the tumor-exclusive neoepitope. In certain embodiments, the resulting engineered CD8+ or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of the genome-integrated NeoTCR gene cassette. Through this approach, native levels of NeoTCR expression are observed in unstimulated and antigen-activated T cell states. The NeoTCR Product manufactured for each patient represents a defined dose of autologous CD8+ and/or CD4+ T cells that are precision genome engineered to express a single neoepitope-specific TCR that is cloned from neoepitope-specific CD8+ T cells individually isolated from the peripheral blood of that same patient. NeoTCR Products are non-limiting examples of Cell Products.

A “NeoTCR Viral Product”, as used herein, has the same definition of NeoTCR Product except that the genome engineering is performed using viral mediated methods. NeoTCR Viral Products are non-limiting examples of Cell Products.

As used herein, “TCR” means T cell receptor.

The term “Complete Response”, as used herein, means the disappearance of all signs of cancer in response to treatment. The term “Partial Response”, as used herein, means a decrease in the size of a tumor, or in the extent of cancer in the body, in response to treatment.

As used herein, the term “PE” means R-phycoerythrin.

The term “dextramer”, as used herein, means a multimerized neoepitope-HLA complex that specifically binds to its cognate NeoTCR.

As used herein, the term “persistence” refers to a quality of cells used in adoptive cell therapies, e.g., NeoTCR Cells, which plays a role in determining the outcome of the therapy. Poor persistence of infused cells is a critical challenge in successful cancer adoptive cell therapy and has been shown to be inversely correlated with durable clinical remissions in patients with cancers. Indeed, poor persistence hinders the long-term effector functions of infused cells in vivo and potentially hampers the long-term therapeutic impacts of adoptive cell therapy. Several factors can influence the persistence of adoptively transferred T cells.

As used herein, “endogenous” means a nucleic acid molecule or polypeptide that is normally expressed in a cell or tissue.

As used herein, “exogenous” means a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, “vector” means the discrete elements that are used to introduce heterologous DNA into cells for either expression or replication thereof. As used herein, a vector can be engineered and used for in vivo or in vitro expression of a polypeptide gene product encoded by a coding sequence inserted into the vector. In certain embodiments, the vector comprises a 2A peptide.

“FACS” as used herein means fluorescence-activated cell sorting. “Flow cytometry” and “FACS” are used interchangeably herein.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product are not considered unacceptably toxic.

As used herein, the term “lot release” refers to a mechanism that allows the monitoring of product quality, through review and testing, of the features of a product. Lot release is the process of evaluating each individual lot of a product, e.g., Cell Product, before its release onto the market or for use as therapeutic. In certain embodiments, the lot release of a product disclosed herein, e.g., a NeoTCR Product, requires an amount of gene edited cells above a certain threshold.

A “subject”, “patient”, or an “individual” for purposes of treatment refers to any animal classified as a mammal, including humans, domestic and farm animals, and zoo, sports, or pet animals, such as dogs, horses, cats, cows, etc. In certain embodiments, the subject is a mammal. In certain embodiments, the mammal is a human.

“Treat”, “treatment”, and “treating” are used interchangeably and, as used herein, mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, the NeoTCR Product of the disclosure is used to delay the development of a proliferative disorder (e.g., cancer) or to slow the progression of such disease.

As used herein, the term “therapeutically effective amount” or “effective amount” refers to an amount of an adoptive cell therapy that is sufficient to reduce, inhibit, or abrogate cancer growth. The amount of cells in an adoptive cell therapy that is therapeutically effective or effective may vary depending on the context. An effective amount can be administered in one or more administrations.

As used herein, the term “IHC” or “immunohistochemistry” refers to immunostaining of cells or tissues.

### 2. Adoptive Cell Therapies

The present disclosure provides methods related to the development and improvement of immunotherapies using the transfer of cells that are engineered to target diseased and/or harmful cells. For example, but without any limitation, the cell can express an exogenous TCR or a chimeric antigen receptor (CAR). In certain non-limiting embodiments, the present disclosure provides methods useful for the assessment of adoptive cell therapy. For example, without any limitation, the methods disclosed herein can determine the persistence of an adoptive cell therapy in a subject. In certain embodiments, the methods described herein relate to the development and improvement of cancer immunotherapies that use the transfer of cells that are engineered to react and kill cancer.

As used herein, “adoptive cell therapy” refers to a therapy in which immune cells, e.g., T cells, are infused to a subject to help the body fight diseases, such as cancer. In certain embodiments, the adoptive cell therapy includes T cells taken from a subject's own blood or tumor tissue, expanded ex vivo, and then infused into to the patient to help the immune system fight the cancer. In certain embodiments, the adoptive cell therapy includes cells, e.g., T cells, that are engineered to improve their ability to target a cancer cell. In certain embodiments, the adoptive cell therapy includes cells of the immune system or hematopoietic stem cells. For example, without any limitation, the adoptive cell therapy can include CD8+ cells, CD4+ cells, NK-cells, delta-gamma T-cells, regulatory T-cells, or tumor-infiltrating lymphocytes. In certain embodiments, the adoptive cell therapy includes T cells. In certain embodiments, the adoptive cell therapy includes NK cells. In certain embodiments, the adoptive cell therapy includes Cell Products, described below. In certain embodiments, the adoptive cell therapy includes NeoTCR Products, described in Section 2.2. In certain embodiments, the adoptive cell therapy includes NeoTCR Viral Products, as described below.

### 2.1. Cell Products

In certain non-limiting embodiments, the present disclosure provides methods useful for the assessment of Cell Products. Cell Products include cells that are gene edited using constructs containing one or more 2A peptide. In certain embodiments, Cell Products can be made using non-viral methods. In certain embodiments, Cell Products can be made using viral methods. In certain embodiments, Cell Products include a construct with a gene of interest, e.g., an exogenous TCR, that is inserted into a genome on a construct that further comprises one or more 2A peptide coding sequences. In certain embodiments, the use of the 2A peptide coding sequences enables the expression of multiple proteins within a single open reading frame through co-translational cleavage events. This approach can overcome the problem of uneven expression of different proteins, which is a major hurdle in gene editing.

Cell Products, as described herein, can include gene edited cells that retain a 2A peptide, or a fragment thereof, in the translated product. In certain embodiments, the gene of interest inserted into a genome of a Cell Product retains a 2A peptide, or a fragment thereof, on a flanking end of the protein expressed by the gene of interest. In certain embodiments, the gene of interest inserted into a genome of a Cell Product retains a 2A peptide, or a fragment thereof, on both flanking ends of the protein expressed by the gene of interest.

Cell Products, as described herein, can also include gene edited cells that fully cleave off the 2A peptide from the gene of interest during translation, such that the protein expressed by the gene or genes of interest inserted into a genome do not have all or a portion of the 2A peptide on either of the flanking ends of the protein expressed by the gene(s). In this scenario, the protein expressed by the inserted gene of interest does not contain any non-native epitopes caused by one or more amino acid of the 2A peptide including on either of the flanking ends of the protein expressed by the gene(s).

In certain embodiments, Cell Products, as described herein, include gene edited cells that are edited using viral methods. In certain embodiments, Cell Products, as described herein, include gene edited cells that are edited using non-viral methods.

### 2.2. NeoTCR Products and NeoTCR Viral Products.

There is increasing evidence that suggests that checkpoint inhibitor-responsive solid tumors are more likely to harbor a higher somatic mutational burden (resulting in expression of tumor-exclusive neoantigens), and the tumors exhibit higher CD8 T cell infiltration and/or exhibit pre-existing high PD-L1 tumor expression (Schumacher and Schreiber, Science 348.6230 (2015): 69-74.). Each of these features represents a higher potential for endogenous immunogenicity of these tumors, namely that the immune system in those patients will have likely initiated a significant T cell immune response prior to initiation of checkpoint inhibitor therapy (Lawrence, et al., Nature 499.7457 (2013): 214-218; Tumeh, et al., Nature 515.7528 (2014): 568-571; Wargo, et al., Current opinion in immunology 41 (2016): 23-31). The application of next generation deep sequencing of tumors and immunologic analysis of the endogenous tumor-targeted T cell response provided compelling evidence for the connection between cancer immunotherapy benefit, tumor mutational burden, and a pre-existing population of neoantigen-specific T cells. The neoantigen-specific population of T cells that specifically recognize and kill the tumor cells harboring these tumor-exclusive mutations (neoantigens) are proposed to be the main mediators of effective cancer immunotherapies to trigger clinical benefit (Tran et al., Nature immunology 18.3 (2017): 255-262; Schumacher and Schreiber, Science 348.6230 (2015): 69-74). Adoptive TCR-T cell therapy targeting neoepitopes holds the potential to overcome the limitations described above.

In certain non-limiting embodiments, the present disclosure provides methods useful for the assessment of NeoTCR Products. The NeoTCR Product is a novel adoptive TCR-T cell therapy engineered with autologous NeoTCRs of native sequence, identified, and isolated from the patient's personal intrinsic T cell cancer immune response. Tumor-specific genomic alterations that initially represent founder (truncal) mutations in each patient, including ‘driver’ mutations for cancer pathology, expand in number and diversify over time as ‘branch’ or ‘passenger’ mutations in later stage malignancies. The spectrum of these accumulated tumor-specific mutations represents a unique private signature of targets for immune recognition in each cancer patient (private neoantigens). T cells that target these private and tumor-exclusive neoantigens (neoepitope or neoE-specific T cells) harbor the potential to exclusively target and kill the tumor cells, while ignoring healthy cells that do not express these tumor-specific mutations. In this way, the immune system of each patient engages the tumors and an appropriately scaled intrinsic immune response, when properly leveraged, has been shown to eradicate the tumors.

Since all cancers are driven by underlying founder or truncal mutations, the NeoTCR Product disclosed herein targets truncal neoepitopes and holds the potential for treatment of any patient with cancer. The NeoTCR Product is an adoptive personalized cell therapy. In certain embodiments, the NeoTCR Product involves engineering an individual's own CD8 and CD4 T cells to express naturally occurring NeoTCRs that already recognize tumor-exclusive neoantigens. These NeoTCRs, therefore, are of native sequence and are derived from pre-existing mutation-targeted CD8 T cells. The NeoTCRs are captured from peripheral blood by a proprietary isolation technology, which authenticates the tumor-exclusive neoepitope targets in each patient.

In certain non-limiting embodiments, the manufacturing process of the NeoTCR Products includes genome engineering of freshly derived CD4 and/or CD8 T cells from a leukopak of the same patient. In certain embodiments, the NeoTCR Products are precision genome engineered to express one NeoTCR in a manner that reconstitutes ‘native’ autologous T cell function and that has been validated to interact with the autologous patient predicted antigens throughout the selection process. The clinical benefit to participants with cancer thus stems from delivering a single dose of ex vivo engineered, tumor mutation-targeted autologous NeoTCR cells, thus providing the potential to trigger rapid and durable responses in patients, some of which have no curative treatment options.

The pharmacological evaluation of the NeoTCR Products and NeoTCR Viral Products demonstrated that NeoTCR cells produced with ex vivo manufacturing processes have potent antigen-specific killing, effector cytokine secretion, and proliferative activity on contact with cognate neoantigen-expressing tumor cells. Furthermore, the NeoTCR Products and NeoTCR Viral Products have been shown to respond to target tumor cells with a strong polyfunctional effector protein secretion response, as demonstrated by bulk T cell and single-cell secretome analysis. The observed polyfunctional T cell effector phenotype is predicted to contribute to the potential for clinical benefit upon infusion of NeoTCR Products and NeoTCR Viral Products into patients with cancer in a manner similar to that observed with polyfunctional CAR-T cells infused into patients with hematologic malignancies.

In certain embodiments, the NeoTCR Products described herein include memory stem cell (T_{MSC}) and central memory (T_{CM}) T cell phenotypes as a result of the ex vivo manufacturing process described herein. These ‘younger’ or less-differentiated T cell phenotypes are described to confer improved engraftment potential and prolonged persistence post-infusion in mouse models and in clinical trials of engineered CAR-T cells in patients with hematologic malignancies. Thus, the administration of NeoTCR Products, including ‘younger’ T cell phenotypes has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Products described herein that were manufactured starting from T cells of patients with cancer. Comparable gene editing efficiencies and functional activities, as measured by antigen-specificity of T cell killing activity, proliferation, and cytokine production, were observed demonstrating that the manufacturing process described herein is successful in generating product with T cells from patients with cancer as starting material.

In certain embodiments, the manufacturing process of the NeoTCR Products disclosed herein in Examples 1 and 2 involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Products or comparable cell products from healthy donors were assessed for cleavage of the candidate off-target sites by deep sequencing, supporting the published evidence that the selected nucleases are highly specific. Further aspects of the precision genome engineering process of the NeoTCR Products described in Examples 1 and 2 herein have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

Accordingly, NeoTCR Products and NeoTCR Nonviral Products provide a novel development in cancer therapy for patients in need thereof. Additional information of the NeoTCR Products can be found in International Patent Publication No. WO2019089610, the content of which is incorporated herein by reference.

### 3. 2A Self-Cleaving Peptides

In certain non-limiting embodiments, the present disclosure provides methods useful for the assessment of adoptive cell therapies, e.g., NeoTCR Products, by determining the expression of a 2A peptide, or a fragment thereof “2A peptides” are a class of 18-22 amino acid long peptides, viral, and self-cleaving. 2A peptides are able to mediate cleavage of peptides during translation in eukaryotic cells. Four well-known members of the 2A peptide class are T2A, P2A, E2A, and F2A. The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus. The self-cleaving mechanism of the 2A peptides is a result of ribosome skipping the formation of a glycyl-prolyl peptide bond at the C-terminus of the 2A. Specifically, the 2A peptides have a C-terminal conserved sequence that is necessary for the creation of steric hindrance and ribosome skipping. In certain embodiments, the C-terminal conserved sequence is GDVEXNPGP (SEQ ID NO: 1). In certain embodiments, the C-terminal conserved sequence is GDVESNPG (SEQ ID NO: 2). The ribosome skipping can result in one of three options: 1) successful skipping and recommencement of translation resulting in two cleaved proteins (the upstream of the 2A protein which is attached to the complete 2A peptide except for the C-terminal proline and the downstream of the 2A protein which is attached to one proline at the N-terminal; 2) successful skipping but ribosome fall-off that results in discontinued translation and only the protein upstream of the 2A; or 3) unsuccessful skipping and continued translation (i.e., a fusion protein). The term “2A” is reference to a specific region of the viral genome. The members of the 2A peptide class are named after the virus from which they were first described.

As used herein, the terms “2A” and “2A peptide” are used interchangeably and mean a class of 18-22 amino acid long, viral, self-cleaving peptides that are able to mediate cleavage of peptides during translation in eukaryotic cells.

### 4. Methods of Use

Although adoptive cell therapies, e.g., NeoTCR Products, have shown clinical success, several challenges remain in enhancing that success. For example, clinical non-responsiveness or relapse has been observed in patients treated with adoptive cell therapies. Accurate and reproducible detection of effective adoptive cell therapies are required to address these challenges and to improve the therapeutic approach of patients with cancer.

During manufacturing of adoptive cell therapies, T cells are transduced or transfected with a vector expressing an antigen binding receptor, e.g., an exogenous TCR, that could integrate into the genome. The detection of the integrated antigen binding receptor can require genomic or transriptomic analysis, e.g., quantitative PCR or digital PCR. However, these methods either cannot be used or are inefficient in adoptive cell therapies that are engineered to express a native sequence of an exogenous T cell receptors (TCR), e.g., NeoTCR Products.

The present disclosure provides methods for detecting kinetics, persistence, biodistribution, and effector functions of adoptive cell therapies. In certain embodiments, the adoptive cell therapy comprises a Cell Product. In certain embodiments, the adoptive cell therapy comprises a NeoTCR Product. In certain embodiments, the adoptive cell therapy comprises a NeoTCR Viral Product. In certain embodiments, the methods disclosed herein can be used for diagnostic use. In certain embodiments, the methods disclosed herein can be used for prognostic use. In certain embodiments, the methods disclosed herein can be used for improving manufacturing processes. In certain embodiments, the methods disclosed herein can be used for clinical trial laboratory operations. In certain embodiments, the methods disclosed herein reduce false negative incidence. In certain embodiments, the methods disclosed herein reduce false positive incidence. In certain embodiments, the methods disclosed herein increase replicability of manufacturing processes.

### 4.1 Diagnostic and Prognostic Methods

Embodiments of the present disclosure relate to methods for diagnosis of presence of an adoptive cell therapy in a subject that has received the adoptive cell therapy. In certain embodiments, the methods include detecting an expression level of a 2A peptide, or a fragment thereof, in a sample from the subject. In certain embodiments, the adoptive cell therapy comprises a cell expressing a 2A peptide, or a fragment thereof, disclosed herein. In certain embodiments, detection of the 2A peptide, or a fragment thereof, is associated with the presence, persistence, and/or expansion of the adoptive cell therapy, depending on the timing and/or amount of 2A protein detected. In certain embodiments, absence of the 2A peptide, or a fragment thereof, is associated with inefficiency of the adoptive cell therapy.

In certain non-limiting embodiments, the present disclosure provides methods for prognosis of a subject treated with an adoptive cell therapy. In certain embodiments, the methods include detecting an expression level of a 2A peptide, or a fragment thereof, in a sample from the subject. In certain embodiments, the adoptive cell therapy comprises a cell expressing a 2A peptide, or a fragment thereof, disclosed herein. In certain embodiments, detection of the 2A peptide, or a fragment thereof, is associated with efficacy of the adoptive cell therapy. In certain embodiments, absence of the 2A peptide, or a fragment thereof, is associated with reduced efficacy of the adoptive cell therapy.

In certain embodiments, the detection of the 2A peptide, or a fragment thereof, can occurs at any time after the subject has received the adoptive cell therapy. In certain embodiments, the detection of the 2A peptide, or a fragment thereof, occurs 1 day, 2 days, 3 days, 4 days, 5 days, 6 days, 7 days, 14 days, 21 days, 28 days, 2 months, 3 months, 4 months, 5 months, 6 months, 9 months, 12 months, between 15-24 months, and yearly thereafter. In certain embodiments, the detection of the 2A peptide, or a fragment thereof, occurs approximately daily for the first week and thereafter weekly through the end of the first month post-infusion, followed by monthly detection of the 2A peptide, or a fragment thereof, through the end of the sixth month post-infusion. In certain embodiments, the detection of the 2A peptide, or a fragment thereof, occurs approximately 1 day, approximately 2 days, approximately 3 days, approximately 4 days, approximately 5 days, approximately 6 days, approximately 7 days, approximately 14 days, approximately 21 days, approximately 28 days, approximately 2 months, approximately 3 months, approximately 4 months, approximately 5 months, approximately 6 months, approximately 9 months, approximately 12 months, approximately between 15-24 months, and approximately yearly thereafter. In certain embodiments, the detection of the 2A peptide, or a fragment thereof, occurs at one or more of the following time points: approximately 1 day, approximately 2 days, approximately 3 days, approximately 4 days, approximately 5 days, approximately 6 days, approximately 7 days, approximately 14 days, approximately 21 days, approximately 28 days, approximately 2 months, approximately 3 months, approximately 4 months, approximately 5 months, approximately 6 months, approximately 9 months, approximately 12 months, approximately between 15-24 months, and approximately yearly thereafter. In certain embodiments, the detection of the 2A peptide, or a fragment thereof, occurs 10 days, 20 days, 30 days, 45 days, and any time in between after the subject has received the adoptive cell therapy. In certain embodiments, the detection of the 2A peptide, or a fragment thereof, occurs 10 days, 20 days, 30 days, 45 days, and any time in between after the subject has received the adoptive cell therapy. In certain embodiments, the detection of the 2A peptide, or a fragment thereof, occurs 1 day, 10 days, 20 days, 30 days, 45 days, and any time in between after the subject has received the adoptive cell therapy. In certain embodiments, the detection of the 2A peptide, or a fragment thereof, occurs 1 month, 2 months, 3 months, 6 month, 9 months, 12 months, and any time in between after the subject has received the adoptive cell therapy. In certain embodiments, the detection of the 2A peptide, or a fragment thereof, occurs 1 year, 2 years, 3 years, 5 years, 10 years, 15 years, 20 years, and any time in between after the subject has received the adoptive cell therapy.

In certain embodiments, a 2A Diagnostics can be used to determine the efficiency of a viral transduction or a non-viral transfection of cells to make a Cell Product.

In certain embodiments, a 2A Diagnostics can be used to determine the presence and/or persistence of a Cell Product in a patient after infusion. Determining the presence and/or persistence of a Cell Product in a patient after infusion is critical in determining efficacy of a Cell Product because if the Cell Product is not present and does not persist in vivo in the patient, it cannot treat the target disease.

In certain embodiments, a 2A Diagnostics can be used to determine the prognosis of a patient after infusion of a Cell Product based on detection of the presence and/or persistence of a Cell Product in a patient after infusion. In certain embodiments, the presence and/or persistence of a Cell Product in a patient after infusion is indicative of a positive prognosis. In certain embodiments, a positive prognosis is indicative of a reduced disease state. In certain embodiments, a positive prognosis is indicative of a stable disease state. In certain embodiments, a positive prognosis is indicative of the ablation or substantial reduction of a disease state. In certain embodiments the disease state is cancer. In cancer, a positive prognosis is stable disease, reduced tumor burden (partial response), or complete response to the Cell Product. In certain embodiments, no detection of the Cell Product in a patient after infusion is a negative prognosis. In certain embodiments, the negative prognosis is indicative of the spread of disease or indicative of a lack of treatment of the disease. In certain embodiments, the disease is cancer and the negative prognosis is additional tumor burden. In certain embodiments, the kit 2A Diagnostic utilizes methods that are identical or substantially similar to the methods disclosed in Examples 4-7. In certain embodiments, the 2A Diagnostic requires permeabilization of the cells without any fixative prior to or concurrently with staining with an anti-2A peptide antibody. In certain embodiments, the 2A Diagnostic requires no fixation of the cells prior to staining with an anti-2A peptide antibody. In certain embodiments the anti-2A antibody is the 3H4 antibody (Novus Biological, Cat. Nos. NBP2-59627AF594 and NBP2-59627AF647) described in Example 4. In certain embodiments the anti-2A antibody binds the same epitope as the 3H4 antibody described in Example 4. In certain embodiments the anti-2A antibody competes for binding with the 3H4 antibody described in Example 4. In certain embodiments, the anti-2A antibody is ABS31 (Sigma-Aldrich, Cat. No. ABS31). In certain embodiments the anti-2A antibody binds the same epitope as the ABS31. In certain embodiments the anti-2A antibody competes for binding with the ABS31 antibody.

In certain embodiments, the 2A Diagnostics are provided and sold as a kit. In certain embodiments, the kit contains instructions for using the 2A Diagnostic. In certain embodiments, the kit contains instructions for using the 2A Diagnostic that are identical or substantially similar to the methods disclosed in Examples 4-7. In certain embodiments, the kit contains instructions for using the 2A Diagnostic that instruct the user to permeabilize the cells of interest but not fix the cells of interest prior to or concurrently with staining with an anti-2A peptide antibody. In certain embodiments, the kit contains instructions for using the 2A Diagnostic that instruct the user not to fix the cells of interest prior to staining with an anti-2A peptide antibody. In certain embodiments the anti-2A antibody is the 3H4 antibody described in Example 4. In certain embodiments the anti-2A antibody binds the same epitope as the 3H4 antibody described in Example 4. In certain embodiments the anti-2A antibody competes for binding with the 3H4 antibody described in Example 4.

The 2A Diagnostics described herein can be manufactured as a kit or other commercial product for the method of detecting Cell Products.

### 4.2. Methods of Treatment

The present disclosure provides methods for treating a subject that has received an adoptive cell therapy disclosed herein, e.g., a Cell Product, comprising performing one of the diagnostic or prognostic methods disclosed herein and administering a second adoptive cell therapy, when inefficiency of the adoptive cell therapy.

In certain embodiments, the second adoptive cell therapy is a Cell Product. In certain embodiments, the second adoptive cell therapy is a NeoTCR Product. In certain embodiments, the second adoptive cell therapy is a NeoTCR Viral Product. In certain embodiments, an adoptive cell therapy disclosed herein can be used to treat disease. In certain embodiments, the second adoptive cell therapy can be used to treat human disease. For example, but without any limitation, the human disease can be an autoimmune disease, a neurological disease and/or injury (including, but not limited to, Alzheimer's disease, Parkinson's disease, spinal cord and nerve injuries and/or damage), a cancer, an infectious disease, a joint disease (including, but not limited to, rebuilding damaged cartilage in joints), an immune system disease, a cardiovascular disease and/or abnormality, aging, an immune deficiency (including, but not limited to, multiple sclerosis and amyotrophic lateral sclerosis), an allergy, or a genetic disorder.

In certain embodiments, the second adoptive cell therapy disclosed herein can be used to treat proliferative diseases such as cancer. Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, hepatoma, lymphangioendotheliosarcoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the cancer is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed young T cells and compositions comprising thereof can be used for treating and/or preventing blood cancers (e.g., leukemias, lymphomas, and myelomas) or ovarian cancer, which are not amenable to conventional therapeutic interventions.

In certain embodiments, the subject can have an advanced form of disease, in which case the treatment objective can include mitigation or reversal of disease progression, and/or amelioration of side effects. The subject can have a history of the condition, for which they have already been treated, in which case the therapeutic objective will typically include a decrease or delay in the risk of recurrence.

In certain embodiments, the second adoptive cell therapy disclosed herein can be used to treat autoimmune diseases or neurological diseases and injuries. In certain embodiments, the second adoptive cell therapy disclosed herein can be used to treat infectious diseases, joint diseases and injuries, or immune diseases and deficiencies. In certain embodiments, the second adoptive cell therapy can be used to treat cardiovascular disease and abnormalities, aging, immune deficiencies. In certain embodiments, the second adoptive cell therapy can be used to treat allergies or genetic disorders.

### 4.3. Additional Methods

Embodiments of the present disclosure related to methods for determining whether a cell has been edited. In certain embodiments, the methods include transducing or transfecting a cell with a vector encoding an antigen binding receptor and a 2A peptide, or a fragment thereof, and detecting an expression level of the 2A peptide, or a fragment thereof, in the cell. In certain embodiments, detection of the 2A peptide, or a fragment thereof, is associated with efficiency of gene editing. In certain embodiments, absence of the 2A peptide, or a fragment thereof, is associated with inefficiency of gene editing.

In certain embodiments, the antigen binding receptor is a T cell receptor (TCR). In certain embodiments, the vector includes first and second homology arms homologous to first and second target nucleic acid sequences and oriented to facilitate homologous recombination. In certain embodiments, the vector includes a nucleotide sequence encoding the TCR positioned between the first and second homology arms. In certain embodiments, the vector includes a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide. In certain embodiments, the first and second nucleotide sequences encoding the 2A ribosome skipping elements have the same amino acid sequences and are codon-diverged relative to each other.

In certain non-limiting embodiments, the present disclosure provides methods for lot releasing a population of cells for adoptive cell therapy. In certain embodiments, the methods include transducing or transfecting the population of cells with a vector comprising an antigen binding receptor and a 2A peptide, or a fragment thereof, detecting an amount of cells expressing the 2A peptide, or a fragment thereof, and releasing the population of cells for therapeutic use when the amount of cells expressing the 2A peptide, or a fragment thereof, is above a threshold.

In certain embodiments, the antigen binding receptor is a T cell receptor (TCR). In certain embodiments, the vector includes first and second homology arms homologous to first and second target nucleic acid sequences and oriented to facilitate homologous recombination. In certain embodiments, the vector includes a nucleotide sequence encoding the TCR positioned between the first and second homology arms. In certain embodiments, the vector includes a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide. In certain embodiments, the first and second nucleotide sequences encoding the 2A ribosome skipping elements have the same amino acid sequences and are codon-diverged relative to each other. In certain embodiments, the threshold is calculated by the formula:

$$\frac{amount of cells expressing the 2 A peptide}{amount of cells in the population of cell} \times 100 \% .$$

In certain embodiments, the threshold is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%.

### 6. Exemplary Embodiments

In certain embodiments, the present disclosure is directed to methods of determining a presence of an adoptive cell therapy in a subject, comprising: detecting an expression level of a 2A peptide in a sample from the subject that has received the adoptive cell therapy, wherein the adoptive cell therapy comprises a cell expressing the 2A peptide, or a fragment thereof.

In certain embodiments of the methods disclosed herein, detection of the 2A peptide, or a fragment thereof, is associated with the presence of the adoptive cell therapy. In certain embodiments of the methods disclosed herein, absence of the 2A peptide, or a fragment thereof, is associated with inefficiency of the adoptive cell therapy. In certain embodiments of the methods disclosed herein, detecting of the 2A peptide, or a fragment thereof, occurs between 1 day and 30 days after the subject has received the adoptive cell therapy. In certain embodiments of the methods disclosed herein, detecting of the 2A peptide, or fragment thereof, occurs between 1 month and 12 months after the subject has received the adoptive cell therapy. In certain embodiments of the methods disclosed herein, detecting of the 2A peptide or fragment thereof occurs between 1 year and 5 years after the subject has received the adoptive cell therapy.

In certain embodiments, the present disclosure is directed to methods of monitoring the persistence of an adoptive cell therapy in a subject that has received the adoptive cell therapy, comprising: detecting an expression level of a 2A peptide, or fragment thereof, in a sample from a first time point from the subject; and detecting the expression level of the 2A peptide, or fragment thereof, in a sample from a second time point from the subject; wherein the adoptive cell therapy comprises a cell expressing the 2A peptide, and wherein detection of the 2A peptide, or fragment thereof, in the sample of the second time point is associated with the persistence of the adoptive cell therapy.

In certain embodiments, the present disclosure is directed to methods of monitoring the persistence of an adoptive cell therapy in a subject that has received the adoptive cell therapy, comprising: detecting an expression level of a 2A peptide, or fragment thereof, in a sample from a first time point from the subject; and detecting the expression level of the 2A peptide, or fragment thereof, in a sample from a second time point from the subject; wherein the adoptive cell therapy comprises a cell expressing the 2A peptide, and wherein absence of the 2A peptide, or fragment thereof, in the sample of the second time point is associated with the inefficiency of the adoptive cell therapy. In certain embodiments of the methods disclosed herein, the methods further comprise administering a second adoptive cell therapy to the subject.

In certain embodiments, the present disclosure is directed to methods of monitoring the expansion of an adoptive cell therapy in a subject that has received the adoptive cell therapy, comprising: detecting an amount of cells expressing a 2A peptide, or fragment thereof, in a sample from a first time point from the subject; and detecting an amount of cells expressing a 2A peptide, or fragment thereof, in a sample from a second time point from the subject; wherein expansion of the adoptive cell therapy occurs when the amount of cells expressing a 2A peptide, or fragment thereof, in a sample of the second time point is increased compared to the amount of cells expressing a 2A peptide or fragment thereof in a sample of the first time point.

In certain embodiments, the present disclosure is directed to methods method of monitoring the expansion of an adoptive cell therapy in a subject that has received the adoptive cell therapy, comprising: detecting an amount of cells expressing a 2A peptide or fragment thereof in a sample from a first time point from the subject; and detecting an amount of cells expressing a 2A peptide or fragment thereof in a sample from a second time point from the subject; wherein no expansion of the adoptive cell therapy occurs when the amount of cells expressing a 2A peptide or fragment thereof in a sample of the second time point is decreased or the same compared to the amount of cells expressing a 2A peptide or fragment thereof in a sample of the first time point.

In certain embodiments of the methods disclosed herein, the second time point occurs between 1 day and 30 days after the first time point. In certain embodiments of the methods disclosed herein, the second time point occurs between 1 month and 12 months after the first time point. In certain embodiments of the methods disclosed herein, the second time point occurs between 1 year and 5 years after the first time point.

In certain embodiments of the methods disclosed herein, the cell is a Cell Product. In certain embodiments of the methods disclosed herein, the cell is a NeoTCR Product. In certain embodiments of the methods disclosed herein, the sample is a blood sample. In certain embodiments of the methods disclosed herein, the sample is a tumor sample. In certain embodiments of the methods disclosed herein, the subject is human. In certain embodiments of the methods disclosed herein, the subject has a cancer. In certain embodiments of the methods disclosed herein, the 2A peptide, or fragment thereof, is detected by flow cytometry. In certain embodiments of the methods disclosed herein, the 2A peptide, or fragment thereof, is detected by immunohistochemistry (IHC). In certain embodiments of the methods disclosed herein, the IHC is ChipCytometry.

In certain embodiments, the present disclosure is directed to methods for the prognosis of a subject treated with an adoptive cell therapy, comprising: detecting the expression level of a 2A peptide, or fragment thereof, in a sample from the subject wherein the adoptive cell therapy comprises a cell expressing the 2A peptide, or fragment thereof, and determining the expression level of the 2A peptide, or fragment thereof, in a sample from the subject, wherein detection of the 2A peptide, or fragment thereof, is associated with efficacy of the adoptive cell therapy. In certain embodiments, the present disclosure is directed to methods for the prognosis of a subject treated with an adoptive cell therapy, comprising: detecting the expression level of a 2A peptide, or fragment thereof, in a sample from the subject wherein the adoptive cell therapy comprises a cell expressing the 2A peptide, or fragment thereof, and determining an expression level of a 2A peptide, or fragment thereof, in a sample from the subject, and wherein no detection of the 2A peptide, or fragment thereof, is associated with reduced efficacy of the adoptive cell therapy.

In certain embodiments of the methods disclosed herein, the methods further comprise administering a second adoptive cell therapy to the subject. In certain embodiments of the methods disclosed herein, detecting of the 2A peptide, or fragment thereof, occurs between 1 day and 30 days after the subject has received the adoptive cell therapy. In certain embodiments of the methods disclosed herein, detecting of the 2A, or fragment thereof, peptide occurs between 1 month and 12 months after the subject has received the adoptive cell therapy. In certain embodiments of the methods disclosed herein, detecting of the 2A, or fragment thereof, peptide occurs between 1 year and 5 years after the subject has received the adoptive cell therapy.

In certain embodiments of the methods disclosed herein, the cell is a Cell Product. In certain embodiments of the methods disclosed herein, the cell is a NeoTCR Product. In certain embodiments of the methods disclosed herein, the sample is a blood sample. In certain embodiments of the methods disclosed herein, the sample is a tumor sample. In certain embodiments of the methods disclosed herein, the subject is human. In certain embodiments of the methods disclosed herein, the subject has a cancer. In certain embodiments of the methods disclosed herein, the 2A peptide, or fragment thereof, is detected by flow cytometry. In certain embodiments of the methods disclosed herein, the 2A peptide, or fragment thereof, is detected by IHC. In certain embodiments of the methods disclosed herein, the IHC is ChipCytometry.

In certain embodiments, the present disclosure is directed to methods of treating a cancer in a subject that has received an adoptive cell therapy, wherein the adoptive cell therapy comprises a cell expressing a 2A peptide, or fragment thereof, the method comprising: detecting the expression level of the 2A peptide, or fragment thereof, in a sample from the subject, wherein absence or decreased detection of the 2A peptide, or fragment thereof, is associated with inefficiency of the adoptive cell therapy; and administering a second adoptive cell therapy, when the expression level of the 2A peptide is absent or decreased.

In certain embodiments of the methods disclosed herein, detecting of the 2A peptide, or fragment thereof, occurs between 1 day and 30 days after the subject has received the adoptive cell therapy. In certain embodiments of the methods disclosed herein, detecting of the 2A peptide, or fragment thereof, occurs between 1 month and 12 months after the subject has received the adoptive cell therapy. In certain embodiments of the methods disclosed herein, detecting of the 2A peptide, or fragment thereof, occurs between 1 year and 5 years after the subject has received the adoptive cell therapy.

In certain embodiments of the methods disclosed herein, the cell is a Cell Product. In certain embodiments of the methods disclosed herein, the cell is a NeoTCR Product. In certain embodiments of the methods disclosed herein, the sample is a blood sample. In certain embodiments of the methods disclosed herein, the sample is a tumor sample. In certain embodiments of the methods disclosed herein, the subject is human. In certain embodiments of the methods disclosed herein, the 2A peptide, or fragment thereof, is detected by flow cytometry. In certain embodiments of the methods disclosed herein, the 2A peptide or fragment thereof is detected by IHC. In certain embodiments of the methods disclosed herein, the IHC is ChipCytometry.

In certain embodiments of the methods disclosed herein, the 2A peptide, or fragment thereof, is a cleaved 2A peptide. In certain embodiments of the methods disclosed herein, the 2A peptide is selected from the group comprising P2A, T2A, E2A, and F2A peptides. In certain embodiments of the methods disclosed herein, the 2A peptide is a P2A or T2A peptide. In certain embodiments of the methods disclosed herein, the 2A peptide is a P2A peptide.

In certain embodiments of the methods disclosed herein, the 2A peptide, or fragment thereof, is detected using an anti-2A antibody or antigen binding portion thereof. In certain embodiments of the methods disclosed herein, the anti-2A antibody cross-competes for binding to the 2A peptide with a reference antibody or antigen-binding portion thereof, wherein the reference antibody or antigen-binding portion is a 3H4 antibody. In certain embodiments of the methods disclosed herein, the anti-2A antibody binds to the same epitope of the 2A peptide with a reference antibody or antigen-binding portion thereof, wherein the reference antibody or antigen-binding portion thereof, wherein the reference antibody or antigen-binding portion is a 3H4 antibody. In certain embodiments of the methods disclosed herein, the anti-2A antibody cross-competes for binding to the 2A peptide with a reference antibody or antigen-binding portion thereof, wherein the reference antibody or antigen-binding portion is an ABS31 antibody. In certain embodiments of the methods disclosed herein, the anti-2A antibody binds to the same epitope of the 2A peptide with a reference antibody or antigen-binding portion thereof, wherein the reference antibody or antigen-binding portion thereof, wherein the reference antibody or antigen-binding portion is an ABS31 antibody. In certain embodiments of the methods disclosed herein, the anti-2A antibody or antigen-binding portion thereof is a 3H4 antibody. In certain embodiments of the methods disclosed herein, the anti-2A antibody or antigen-binding portion thereof is an ABS31 antibody. In certain embodiments of the methods disclosed herein, the anti-2A antibody binds to an epitope having an amino acid sequence set forth in SEQ ID NO: 3.

In certain embodiments, the present disclosure is directed to methods of determining whether a cell has been edited, comprising transducing or transfecting a cell with a vector encoding an antigen binding receptor and a 2A peptide; detecting an expression level of the 2A peptide, or fragment thereof, in the cell; wherein detection of the 2A peptide, or fragment thereof, is associated with efficiency of gene editing. In certain embodiments, the present disclosure is directed to methods of determining whether a cell has been edited, comprising transducing or transfecting a cell with a vector encoding an antigen binding receptor and a 2A peptide; detecting an expression level of the 2A peptide, or fragment thereof, in the cell; wherein absence of the 2A peptide, or fragment thereof, is associated with inefficiency of gene editing.

In certain embodiments of the methods disclosed herein, the antigen binding receptor is a T cell receptor (TCR). In certain embodiments of the methods disclosed herein, the vector comprises: first and second homology arms homologous to first and second target nucleic acid sequences and oriented to facilitate homologous recombination; a nucleotide sequence encoding the TCR positioned between the first and second homology arms; and a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide, wherein the first and second nucleotide sequences encoding the 2A ribosome skipping elements have the same amino acid sequences and are codon-diverged relative to each other.

In certain embodiments, the present disclosure is directed to methods of lot releasing a population of cells for adoptive cell therapy, comprising: transducing or transfecting the population of cells with a vector comprising an antigen binding receptor and a 2A peptide; detecting an amount of cells expressing the 2A peptide, or fragment thereof; releasing the population of cells for therapeutic use when the amount of cells expressing the 2A peptide is above a threshold.

In certain embodiments of the methods disclosed herein, the antigen binding receptor is a T cell receptor (TCR). In certain embodiments of the methods disclosed herein, the vector comprises: first and second homology arms homologous to first and second target nucleic acid sequences and oriented to facilitate homologous recombination; a nucleotide sequence encoding the TCR positioned between the first and second homology arms; and a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide, wherein the first and second nucleotide sequences encoding the 2A ribosome skipping elements have the same amino acid sequences and are codon-diverged relative to each other. In certain embodiments of the methods disclosed herein, the threshold is calculated by the formula:

$$\frac{amount of cells expressing the 2 A peptide}{amount of cells in the population of cell} \times 100 \% .$$

In certain embodiments of the methods disclosed herein, the threshold is equal to or greater than 5%. In certain embodiments of the methods disclosed herein, the population of cell comprises a Cell Product. In certain embodiments of the methods disclosed herein, the population of cell comprises a NeoTCR Product. In certain embodiments of the methods disclosed herein, the 2A peptide, or fragment thereof, is detected by flow cytometry. In certain embodiments of the methods disclosed herein, the 2A peptide, or fragment thereof, is detected by IHC. In certain embodiments of the methods disclosed herein, the IHC is ChipCytometry.

In certain embodiments of the methods disclosed herein, the 2A peptide is a cleaved 2A peptide. In certain embodiments of the methods disclosed herein, the 2A peptide is selected from the group comprising P2A, T2A, E2A, and F2A peptides. In certain embodiments of the methods disclosed herein, the 2A peptide is P2A or T2A. In certain embodiments of the methods disclosed herein, the 2A peptide is P2A.

In certain embodiments of the methods disclosed herein, the 2A peptide, or fragment thereof, is detected using an anti-2A antibody or antigen binding portion thereof. In certain embodiments of the methods disclosed herein, the anti-2A antibody cross-competes for binding to the 2A peptide with a reference antibody or antigen-binding portion thereof, wherein the reference antibody or antigen-binding portion is a 3H4 antibody. In certain embodiments of the methods disclosed herein, the anti-2A antibody binds to the same epitope of the 2A peptide with a reference antibody or antigen-binding portion thereof, wherein the reference antibody or antigen-binding portion thereof, wherein the reference antibody or antigen-binding portion is a 3H4 antibody. In certain embodiments of the methods disclosed herein, the anti-2A antibody cross-competes for binding to the 2A peptide with a reference antibody or antigen-binding portion thereof, wherein the reference antibody or antigen-binding portion is an ABS31 antibody. In certain embodiments of the methods disclosed herein, the anti-2A antibody binds to the same epitope of the 2A peptide with a reference antibody or antigen-binding portion thereof, wherein the reference antibody or antigen-binding portion thereof, wherein the reference antibody or antigen-binding portion is an ABS31 antibody. In certain embodiments of the methods disclosed herein, the anti-2A antibody or antigen-binding portion thereof is a 3H4 antibody. In certain embodiments of the methods disclosed herein, the anti-2A antibody or antigen-binding portion thereof is an ABS31 antibody. In certain embodiments of the methods disclosed herein, the anti-2A antibody binds to an epitope having an amino acid sequence set forth in SEQ ID NO: 3.

In certain embodiments, the present disclosure is directed to methods of treating a patient with a Cell Product, the method comprising: administering a patient in need thereof a Cell Product, drawing blood or collecting a tumor sample from the patient at a future time point, and analyzing the blood or tumor sample using FACS or IHC analysis to determine if the 2A peptide or antibody binding fragment thereof is present using an anti-2A antibody, wherein if the 2A peptide or antibody binding fragment thereof is detected then the Cell Product is present and persisting in the patient and if the 2A peptide or antibody binding fragment thereof is not detected then the Cell Product is no longer in the patient, wherein if the Cell Product is present and persisting, the product is effective at implanting into the patient and no additional therapy is administered to the patient, and wherein if the Cell Product is not present, the product is not effective at implanting into the patient and a new therapy is administered to the patient.

In certain embodiments of the methods disclosed herein, the Cell Product is a NeoTCR product. In certain embodiments of the methods disclosed herein, the patient is a patient with cancer. In certain embodiments of the methods disclosed herein, the future time point is selected from a time point between 1-10 days, 11-30 days, 31-90 days, 91-180 days, and 181-365 days. In certain embodiments of the methods disclosed herein, the future time point is selected from a time point between 1-2 years, 2-5 years, 5-10 years, 10-20 years, and 20-50 years.

In certain embodiments, the present disclosure is directed to methods of detecting a cleaved 2A peptide or antibody binding fragment thereof in cells of a Cell Product using FACS, comprising one of the following methods: a) Method 1, comprising: staining the cells with a live/dead label, fixing the cells with a fixative, staining the cells with cell surface markers of interest, staining the cells with a permeabilization buffer with an anti-2A antibody, and fixing the cells with a fixative; b) Method 2 comprising: staining the cells with a live/dead label, staining the cells with cell surface markers of interest, fixing the cells with a fixative, staining the cells with a permeabilization buffer with an anti-2A antibody, and fixing the cells with a fixative; and c) Method 3 comprising: staining the cells with a live/dead label, staining the cells with cell surface markers of interest, staining the cells with a permeabilization buffer with an anti-2A antibody, and fixing the cells with a fixative; wherein the stained cells from Methods 1, 2, or 3 are analyzed on a flow cytometer using FACS analysis. In certain embodiments of the methods disclosed herein, the cells are gene edited using the methods described in Example 2 and/or 3.

In certain embodiments, the present disclosure is directed to methods of determining the prognosis of a patient treated with a Cell Product, the method comprising: administering a patient in need thereof a Cell Product, drawing blood or collecting a tumor sample from the patient at a future time point, and analyzing the blood or tumor sample using FACS or IHC analysis to determine if the 2A peptide or antibody binding fragment thereof is present using an anti-2A antibody, wherein if the 2A peptide or antibody binding fragment thereof is detected then the Cell Product is present and persisting in the patient and if the 2A peptide or antibody binding fragment thereof is not detected then the Cell Product is no longer in the patient, wherein if the Cell Product is present and persisting, the prognosis of the patient is good for responding to the Cell Product therapy, and wherein if the Cell Product is not present, the prognosis of the patient is bad for responding to the Cell Product therapy.

In certain embodiments of the methods disclosed herein, the Cell Product is a NeoTCR product. In certain embodiments of the methods disclosed herein, the patient is a patient with cancer. In certain embodiments of the methods disclosed herein, a good prognosis is partial or complete response to the Cell Therapy. In certain embodiments of the methods disclosed herein, a bad prognosis is no response to the Cell Therapy and/or disease progression.

In certain embodiments, the present disclosure is directed to methods of determining the persistence and/or presence of a Cell Product in a patient after infusion of the Cell Product, the method comprising: drawing blood or collecting a tumor sample from the patient at a time point following administration of the Cell Product, and analyzing the blood or tumor sample using FACS or IHC analysis to determine if the 2A peptide or antibody binding fragment thereof is present using an anti-2A antibody, wherein the Cell Product is present and has persisted in the patient if the 2A peptide or antibody binding fragment thereof is detected using the anti-2A antibody.

In certain embodiments, the present disclosure is directed to methods of determining the expansion of a Cell Product in a patient after infusion of the Cell Product, the method comprising: drawing blood or collecting a tumor sample from the patient at two or more time points following administration of the Cell Product, analyzing the blood or tumor samples using FACS or IHC analysis to determine if the 2A peptide or antibody binding fragment thereof is present using an anti-2A antibody, and determining the difference between the number of cells stained with the anti-2A antibody at the two or more timepoints, wherein, an increase in the cell numbers stained with the anti-2A antibody between a first time point and a second is evidence of cell expansion, and wherein a decrease or no increase in the cell numbers stained with the anti-2A antibody between a first time point and a second is evidence of no cell expansion.

In certain embodiments, the present disclosure is directed to methods for detecting the rate of gene editing of a gene editing reaction, the method comprising: permeabilizing and staining the cells from the gene editing reaction with an anti-2A peptide antibody, sorting the cells using FACS or IHC to identify the number of unstained and stained cells, and calculating the rate of gene editing using the following calculation: (number of cells positive for anti-2A antibody staining)/(total number of cells). In certain embodiments of the methods disclosed herein, the permeabilizing and staining the cells is selected from the group consisting of Method 1, Method 2, and Method 3 disclosed herein.

In certain embodiments, the present disclosure is directed to methods for qualifying a Cell Product for release for the treatment of patients in need thereof, the method comprising: taking a sample of the Cell Product, analyzing the sample using FACS or IHC analysis to determine if the 2A peptide or antibody binding fragment thereof is present using an anti-2A antibody, and determining the percent of edited cells, wherein, the Cell Product is released for the treatment of patients if the percent of edited cells meets the product specification for release.

In certain embodiments of the methods disclosed herein, the percent of edited cells is determined using the following formula: (number of cells positive for anti-2A antibody staining)/(total number of cells). In certain embodiments of the methods disclosed herein, the product specification for release is at least 5% gene edited cells. In certain embodiments of the methods disclosed herein, the future time point is selected from a time point between 1-10 days, 11-30 days, 31-90 days, 91-180 days, and 181-365 days. In certain embodiments of the methods disclosed herein, the future time point is selected from a time point between 1-2 years, 2-5 years, 5-10 years, 10-20 years, and 20-50 years. In certain embodiments of the methods disclosed herein, the anti-2A antibody binds to the epitope DVEENPG (SEQ IN NO: 3). In certain embodiments of the methods disclosed herein, the anti-2A antibody is the 3H4 or the ABS31 antibody. In certain embodiments of the methods disclosed herein, the anti-2A antibody competes for binding with the 3H4 and/or ABS31 antibody. In certain embodiments of the methods disclosed herein, the anti-2A antibody binds the same epitope as the 3H4 and/or ABS31 antibody. In certain embodiments of the methods disclosed herein, the IHC is ChipCytometry.

### EXAMPLES

The following are examples of methods and compositions of the invention. It is understood that various other embodiments may be practiced, given the general description provided above.

### Example 1. Insertion of Genes into an Endogenous Locus

Constructs containing genes of interest were inserted into endogenous loci. This was accomplished with the use of homologous repair templates containing the coding sequence of the gene of interest flanked by left and right HR arms. In addition to the HR arms, the gene of interest is sandwiched between 2A peptides, a protease cleavage site that is upstream of the 2A peptide to remove the 2A peptide from the upstream translated gene of interest, and signal sequences (FIG. 2A). Once integrated into the genome, the gene of interest was transcribed as single messenger RNA. During translation of this gene of interest messenger RNA, the flanking regions are unlinked from the gene of interest by the self-cleaving 2A peptide and the protease cleavage site was cleaved for the removal of the 2A peptide upstream from the translated gene of interest (FIG. 2B). In addition to the 2A peptide and protease cleavage site, a gly-ser-gly (GSG) linker can be inserted before each 2A peptide to further enhance the separation of the gene of interest from the other elements in the expression cassette.

It was determined that P2A peptides were superior to other 2A peptides for Cell Products because of its efficient cleavage. Accordingly, two (2) P2A peptides and codon divergence were used in order to express the gene of interest without introducing any exogenous epitopes from remaining amino acids on either end of the gene of interest from the P2A peptide (by use of a protease cleavage site) and the P2A peptide was specifically selected as the 2A peptide of choice in combination with codon divergence to avoid having tandem repeats that would lead to genomic instability and deletions (the codon divergence element). The benefit of the gene edited cell having no exogenous epitopes (i.e., no flanking P2A peptide amino acids on either side of the gene of interest) is that 1) immunogenicity is decreased, 2) there is less likelihood of a patient infused with a Cell Product containing the gene edited cell to have an immune reaction against the gene edited cell, and 3) to increase the chance of neoTCR to function normally in the gene edited cell because there are no N or C terminal fusion.

### Example 2. NeoTCR Integration in TCRα Locus Using 2A Peptides for the Preparation of a NeoTCR Product

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to expression of only TCR sequences encoded by the NeoTCR construct. The general strategy was applied using circular HR templates as well as with linear templates.

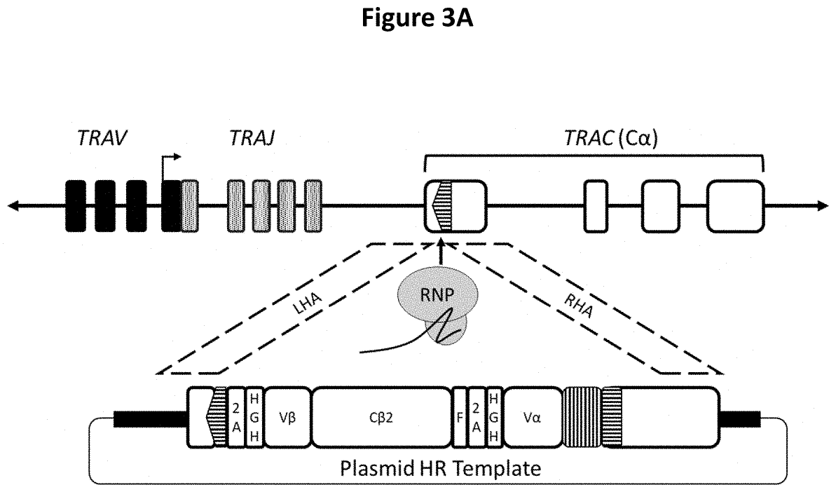

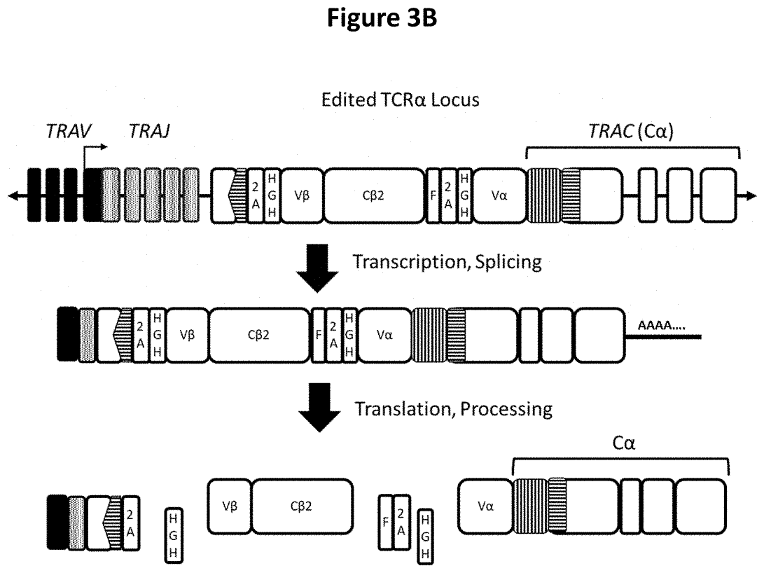

The neoantigen-specific TCR construct design is diagrammed in FIGS. 3A and 3B. The target TCRα locus (Cα) is shown along with the plasmid HR template, and the resulting edited sequence and downstream mRNA/protein products are shown. The target TCRα locus (endogenous TRAC) and its CRISPR Cas9 target site (horizontal stripe, cleavage site designated by arrow) are shown (FIG. 3A). The circular plasmid HR template with the polynucleotide encoding the NeoTCR, which is located between left and right homology arms (“LHA” and “RHA” respectively), is shown (FIG. 3A). The region of the TRAC introduced by the HR template that was codon optimized is shown (vertical stripe). The TCRβ constant domain was derived from TRBC2, which is indicated as being functionally equivalent to TRBC1. Other elements in the NeoTCR cassette include: 2A=P2A ribosome skipping element; F=furin cleavage site upstream of 2A that removes the 2A tag from the upstream TCRβ protein; HGH=human growth hormone signal sequence. The HR template of the NeoTCR expression gene cassette includes two flanking homology arms to direct insertion into the TCRα genomic locus targeted by the CRISPR Cas9 nuclease RNP with the TCRα guide RNA. These homology arms (LHA and RHA) flank the neoE-specific TCR sequences of the NeoTCR expression gene cassette. While the protease cleavage site used in this example was a furin protease cleavage site, any appropriate protease cleavage site known to one of skill in the art could be used. Similarly, while HGH was the signal sequence chosen for this example, any signal sequence known to one of skill in the art could selected based on desired trafficking and used.

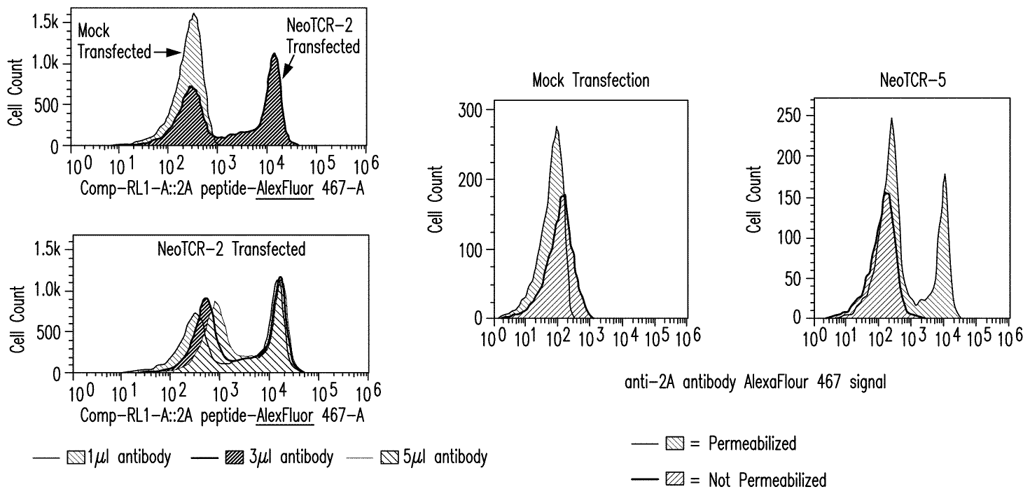

Once integrated into the genome (FIG. 3B), the NeoTCR expression gene cassette is transcribed as a single messenger RNA from the endogenous TCRα promoter, which still includes a portion of the endogenous TCRα polypeptide from that individual T cell (FIG. 3B). During ribosomal polypeptide translation of this single NeoTCR messenger RNA, the NeoTCR sequences are unlinked from the endogenous, CRISPR-disrupted TCRα polypeptide by self-cleavage at a P2A peptide (FIG. 3B). The encoded NeoTCRα and NeoTCRβ polypeptides are also unlinked from each other through cleavage by the endogenous cellular human furin protease and a second self-cleaving P2A sequence motifs included in the NeoTCR expression gene cassette (FIG. 5B). The NeoTCRα and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates removal of the 2A sequence from the upstream TCRβ chain to reduce potential interference with TCRβ function. Inclusion of a gly-ser-gly linker before each 2A (not shown) further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 (vertical stripe) reduces the likelihood of the entire cassette being lost over time through removal of intervening sequence of two direct repeats.

### Example 3. Integration of a Gene of Interest in Genetic Locus Using 2A Peptides for the Preparation of a Cell Product

As described in Example 2 above, 2A peptides can be used in a linear or circular construct to flank both ends of a gene of interest (in the case of Example 2, TCRα) and through non-viral methods (in the case of Example 2, CRISPR/Cas9) insert that gene of interest into the genome of a cell for translation and expression into an active protein.

Similarly, genes of interest can be inserted into genomes using 2A peptides and non-viral methods using any suitable nuclease known to one in the art. For example, these nucleases include but are not limited to zinc finger nucleases (ZFNs), homing endonucleases, TALENs, CRISPR/Cas (a Cas other than Cas9).

Examples of non-nuclease, non-viral methods where 2A peptides can be used for gene editing include but are not limited to gene addition through transposition, including but not limited to Tc1/mariner, PIF/Harbinger, hAT, Mutator, Merlin, Transib, P, piggyBac and CACTA. Recombination including Cre, Flp, PhiC31. Furthermore, any homology-directed repair editing including prime editing can be used.

Furthermore, where appropriate, viral methods of gene editing and gene integration can be used with 2A peptides flanking the gene of interest to be inserted into a genome. Non-limiting examples of viral methods include targeted integration (including but not limited to AAV) and random integration (including but not limited to lentiviral approaches).

Using any of the gene editing methods described above or known to one of skill in the art, appropriate constructs can be designed to include a gene of interest flanked by 2A peptides. Additional functional elements, including but not limited to signal sequences, enzymes, and protease cleavage sites can also be included the constructs for gene editing (including transcription, splicing, translation, and processing of the gene of interest).

Alternatively, using any of the gene editing methods described above or known to one of skill in the art, appropriate constructs can be designed to include one or more gene(s) of interest with one or more 2A peptides included in the constructs to separate the gene(s). Such use of 2A peptides can be used, for example in viral or non-viral gene editing methods. Examples of such viral methods include but are not limited to targeted and non-targeted methods described herein. Additional functional elements, including but not limited to signal sequences, enzymes, and protease cleavage sites can also be included the constructs for gene editing (including transcription, splicing, translation, and processing of the gene of interest).

Constructs can be linear or circular (for example, a plasmid) and double- or single-stranded. An example of a plasmid construct designed to insert a gene of interest into a genome of a cell is provided in FIG. 2A. As shown in FIG. 2A, a gene of interest is flanked by 2A peptide sequences and includes option signal sequences and a protease cleavage site. FIG. 2B shows the translation and processing of the gene of interest once integrated into a genome.

This method of including the 2A peptides on the flanking ends of a gene of interest can be used to make Cell Products with any gene of interest inserted into the genome of a cell.

### Example 4. P2A Peptide Staining by Intracellular Flow Cytometry as a Direct Assessment of Gene Editing Efficiency

As shown in FIGS. 2A, 2B, 3A, and 3B, it is possible to engineer a gene of interest into a specific locus within a specific cell's genome, e.g. a primary cell such as a T cell or an NK cell, without creating non-endogenous epitopes on the transgene resulting from the flanking ends of the 2A peptide used during the engineering steps (i.e., without creating non-native fusions to the delivered genes). Accordingly, the final translated product is the gene of interest without any flanking ends. This is optimal for in vivo use and cell therapies for patient administration because no non-native fusions are created which could result in a cell product with a decreased chance adverse reactions to the product (including but not limited to a decreased chance of anti-drug antibody reactions) and/or a cell product with a neoTCR that has a modified function as a result of the N- and C-terminal fusions; however, without a tag it presents a challenge for efficient and accurate testing to 1) confirm the cells express the gene of interest and 2) confirm that the cells are able to persist and divide in the patient once administered. This problem is further magnified when the gene of interest is a native protein (i.e., a protein that is naturally produced by the patient). This is because detection of the gene of interest translated protein is similar, substantially similar, and/or identical to the naturally occurring gene. Accordingly, in order to detect the gene edited cells in a patient or in a pool of cells based on translated protein, it is necessary to detect a non-native component of the gene edited cell and not the translated protein of the gene of interest itself.

The ability to detect gene edited cells, especially those edited with native sequences, by messenger RNA and PCR is difficult, costly and time consuming and requires equipment that is not readily available in every clinic/hospital. In a clinical setting where time is critical for the good of the patient, a protein-based method of detecting positive gene edited cells is needed. Such a technique is described herein and addresses this unmet need in the world of cell therapies.

Based on this unmet need, an assay was developed to detect the cleaved 2A peptide that remains intracellular while the gene of interest is translated and trafficked to its desired location. For TCRs, the desired location of the gene of interest is the cell membrane.

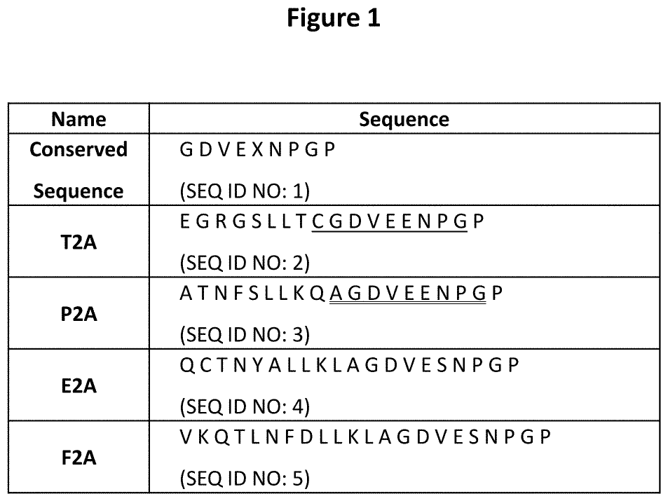

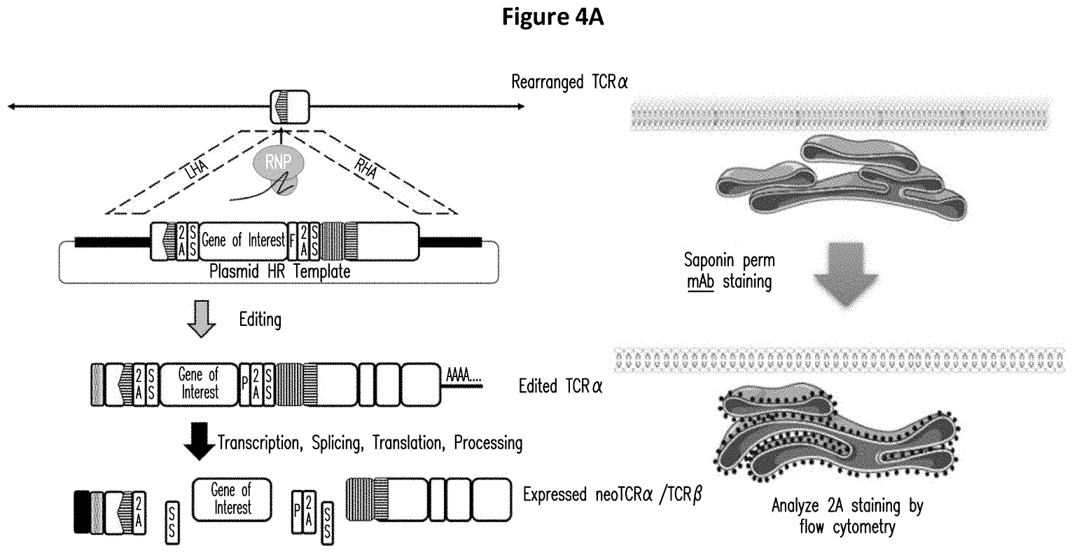

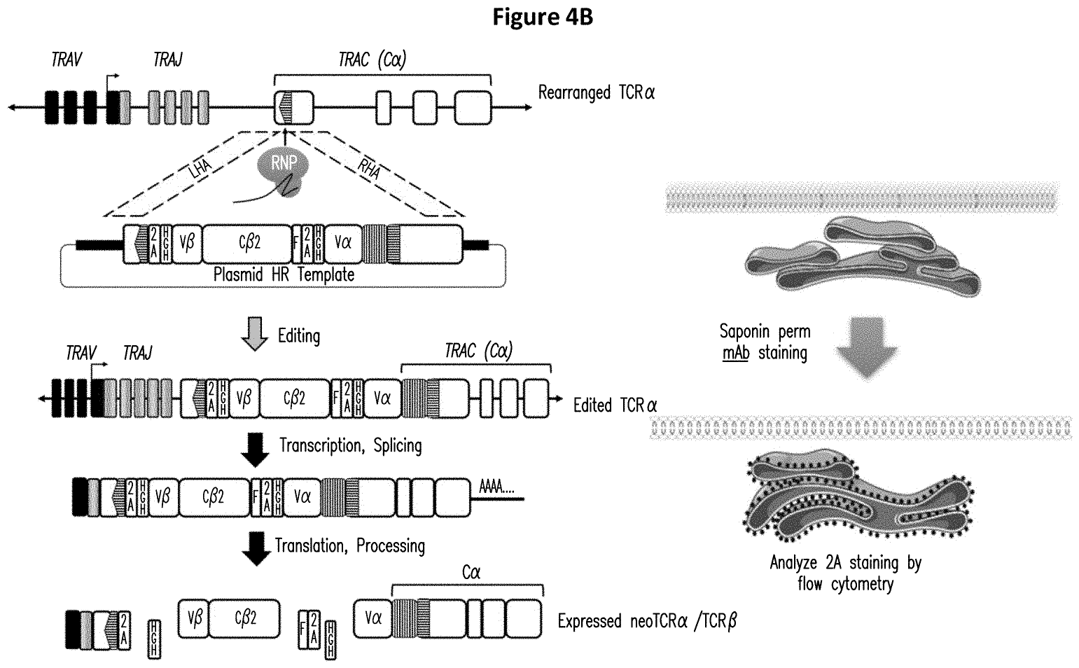

It was not expected for such intracellular detection of the cleaved 2A peptide to be successful. Specifically, the 2A peptides are small peptides (see FIG. 1) and upon cleavage from the protein of interest, they become fragments of the original peptide. Such small peptides and fragments of proteins (i.e., the upstream partial TRA) are expected to go through rapid recycling and degradation within a cell and it was unexpected that the cleaved peptide could be detected in the intracellular portion of a cell with an antibody (see FIG. 4). It was also unexpected and discovered that the truncated NeoTCR with the 2A peptide as shown in FIG. 4 is retained in the endoplasmic reticulum and that minimal 2A peptide was present intracellularly.

2A peptide staining can be readily detected upon cell permeabilization. Following the NeoTCR integration in TCRα locus using 2A peptides non-viral gene editing methods described in Example 2, anti-2A peptide antibody detection of the 2A peptide in the resulting gene edited NeoTCR cells was tested (FIG. 5A). An anti-2A antibody that binds to the DVEENPG (SEQ ID NO: 3) epitope of the 2A peptide was used. This epitope was determined by analyzing the sequence overlap of the T2A, P2A, E2A, and F2A sequences (see bold sequences in FIG. 1). It was determined, based on the P2A cleaving, that it would be desirable to find an anti-P2A antibody that binds to the AGDVEENPG (SEQ ID NO: 4) sequence of the P2A peptide (see, FIG. 1, double underlined sequence of the P2A peptide). As shown in FIG. 1, the single underlined sequence of the T2A peptide represents the epitope recognized by the Novus Biologicals 3H4 monoclonal antibody designed for Western Blot detection of T2A (the “3H4 antibody”) according to the manufacturer. Given that the 3H4 antibody was able to bind T2A and P2A it was determined that the critical epitope for detection with an anti-2A antibody is DVEENPG (SEQ ID NO: 3). The experiments presented herein show that the 3H4 antibody binds to the P2A peptide (FIGS. 5A and 5B).

3H4 antibody labeled with Alexa Fluor 647 was used in this experiment. It was determined that it was necessary to permeabilize the cells for 2A peptide specific staining using FACS (FIG. 5B). Any fluorophore with the correct absorption/emission spectrum could be used for the FACS analysis. This shows that the cleaved 2A peptide is intracellular and that despite rapid turnover and degradation of the cleaved 2A peptide in the cytosol, it is unexpectedly possible to detect the cleaved 2A peptide with an antibody that binds to the 2A peptide. Specifically, it was shown that an antibody that binds to the DVEENPG (SEQ ID NO: 3) sequence of the 2A peptide is able to detect cleaved 2A peptide in the intracellular space of a cell. In summary, the cleaved 2A peptide is not detectable on the cell surface as evidenced by the need to permeabilize the cells to achieve specific staining.

The gates for the FACS analysis shown in FIG. 5B were set on lymphocytes, single cells, that were live, and CD8+. As shown, there was no staining with the 3H4 antibody in unpermeabilized cells and staining when the cells were permeabilized.

The FACS analysis shown in FIG. 5A was gated on live cells that were permeabilized. As shown, mock transfected cells (i.e., not gene edited) did not express any 2A peptide and did not have any 3H4 antibody staining. In contrast, the gene edited cells (the NeoTCR-2 cells) that were stained with either 1, 3, or 5 microliters of 3H4 antibody all showed detection of intracellular 2A in the permeabilized cells.

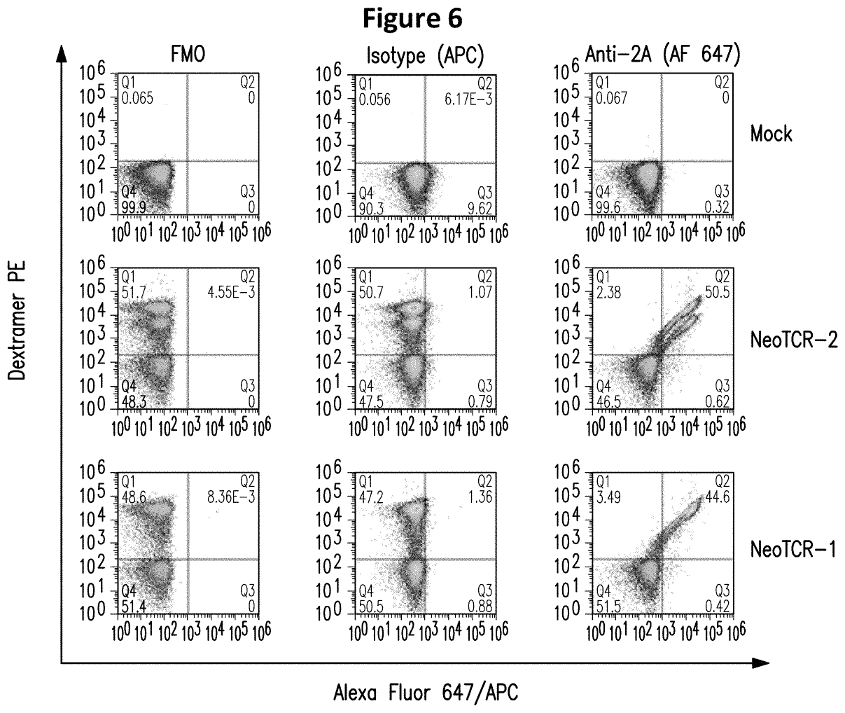

Additional FACS experiments were performed on permeabilized cells to confirm that the 2A staining was specific to gene edited cells. This was done by dual staining: 1) staining for the 2A peptide with 3H4 (Alexa Fluor 647/APC), and 2) binding of PE labeled Dextramer (FIG. 6). Dextramer is a label to detect the NeoTCR that is introduced to the cell using the gene editing methods described herein. The 3H4 antibody detects the 2A peptide that is cleaved during translation of the gene edited NeoTCR construct as described herein. As show in the FMO and Isotype (APC) columns, there is no background signal for the 2A peptide which is expected given that the FMO and Isotype columns do not include exposure to the 3H4 antibody. The “Mock” row is a second control. This row is of cells that were not gene edited. As expected, there is no Dextramer signal and no 2A peptide signal. In contrast to the Mock row, the Gene Edited rows 1 and 2 are of cells sorted by FACS that have been gene edited to express a NeoTCR using the methods described herein (including the use of 2A peptides). As shown in the Gene Edited rows 1 (NeoTCR-2) and 2 (NeoTCR-1), the upper right quadrant (Q2) shows significant cells that are positive for both Dextramer (i.e., signal for the NeoTCR) and 2A peptide (i.e., proof of successful gene editing). This finding was unexpected. Specifically, it was unexpected that 1) it would be possible to detect a cleaved 2A peptide using FACS given the size and fast recycling/cellular processing of the cleaved 2A peptide and 2) that FACS was a viable method of detected the cleaved 2A peptide with a monoclonal antibody because scientific literature excluded such use of an antibody and more importantly recommended non-FACS detection methods such as Western Blot where full cell lysate of a pool of cells is used (in contrast to single cell detection by FACS). Even the literature supplied by the manufacturer of the 3H4 antibody did not list FACS as a viable option for 2A peptide detection. It is also unexpected for 2A peptides in particular because flow antibodies must be highly specific as compared to western blot antibodies (where noise can be ignored based on size). The 2A peptide sequence is very limited in size and sequence from which to derive different antibodies (not many epitopes/options). It was thus expected that the few clones generate would not be suitable for flow cytometry and the fact that it was found that 3H4 worked was surprising and unexpected.

TABLE 1
Gene edited cells used in Examples

| Reagent ID | Reagent | Description |
|---|---|---|
| NeoTCR-1 | Cells | Gene edited T cells from patient 1 with a |
| | | NeoTCR A |
| NeoTCR-2 | Cells | Gene edited T cells from patient 1 with a |
| | | NeoTCR B |
| NeoTCR-3 | Cells | Gene edited T cells from patient 1 with a |
| | | NeoTCR C |
| NeoTCR-4 | Cells | Gene edited T cells from patient 1 with a |
| | | NeoTCR D |
| NeoTCR-5 | Cells | Gene edited T cells from patient 1 with a |
| | | NeoTCR E |
| NeoTCR-6 | Cells | Gene edited T cells from patient 1 with a |
| | | NeoTCR F |
| NeoTCR-7 | Cells | Gene edited T cells from patient 1 with a |
| | | NeoTCR G |
| NeoTCR-8 | Cells | Gene edited T cells from patient 2 with a |
| | | NeoTCR B |

Two of the key reasons why detection of the cleaved 2A peptide is important for the manufacture of NeoTCR products are 1) certain NeoTCRs are not strong binders of dextramer, and 2) dextramers must be specific to each neoTCR which is expensive and complex for personalized medicines. The fact that NeoTCRs are not strong binders of dextramer can be a byproduct of the CD4 T cell population that are gene edited using the methods, or modifications of the methods, described herein. Specifically, while naturally occurring MHC-I TCRs are presumed to require concurrent CD8 co-receptor help to stabilize peptide-MHC binding, higher affinity TCRs drive CD8-independent target binding and T cell activation. CD4 T cells, when engineered with high affinity NeoTCRs, are thus able to recognize peptide-MHC-I targets and trigger effector T cell functions. However, lower affinity TCRs are dependent on CD8 co-receptors to trigger T cell activation. Accordingly, lower affinity TCRs may not be able to effectively bind to dextramer in the FACS analysis to determine whether or not cells were gene edited.

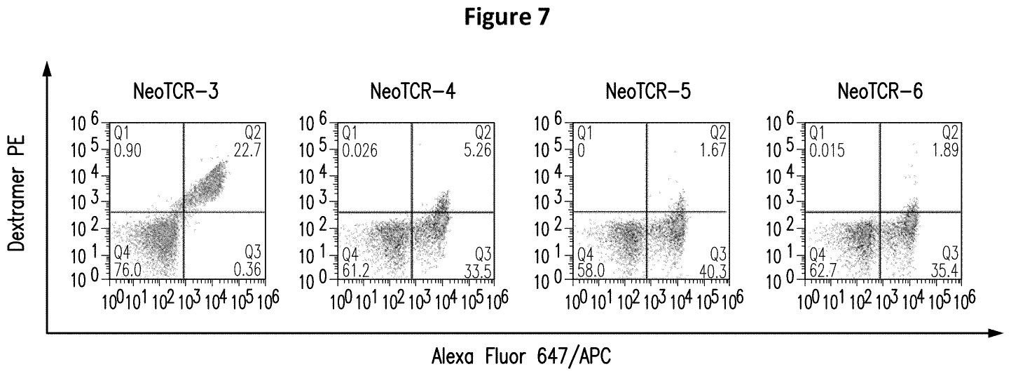

Thus, having the ability to detect gene editing without having to rely on the detection of the insertion of the NeoTCRs by dextramer binding provides a solution to a previously unsolved problem. Experiments were designed to confirm the detection of a cleaved 2A peptide using FACS and an anti-2A peptide antibody in CD4 T cells in the absence of NeoTCR detection with a dextramer (FIG. 7).

FIG. 7 shows gene edited CD4 T cells (FACS gated on lymphocytes, singlets, live cells, and CD4+ cells) from NeoTCR-3, NeoTCR-4, NeoTCR-5, and NeoTCR-6 (see Table 1). For NeoTCR-3, the NeoTCR was detected by dextramer and the cleaved 2A peptide was detected by the 3H4 anti-2A peptide antibody (upper right quadrant (Q2)). This shows that the NeoTCR was a high affinity NeoTCR that can bind dextramer in a CD8 independent fashion. Unlike NeoTCR-3, NeoTCRs 4-6 show very little dextramer binding (upper left quadrant (Q1) and upper right quadrant (Q2)). Notwithstanding the minimal dextramer staining, NeoTCRs 4-6 show substantial detection of the cleaved 2A peptide (lower right quadrant (Q3)). Accordingly, gene editing was confirmed using an anti-2A peptide antibody to detect the cleaved 2A peptide using FACS in low affinity NeoTCRs that benefit from the CD8 co-receptors. Furthermore, 2A peptide stain is independent of dextramer staining, with CD4 T cells that do not stain by dextramer readily staining at similar levels as CD8 T cells that do stain with dextramer.

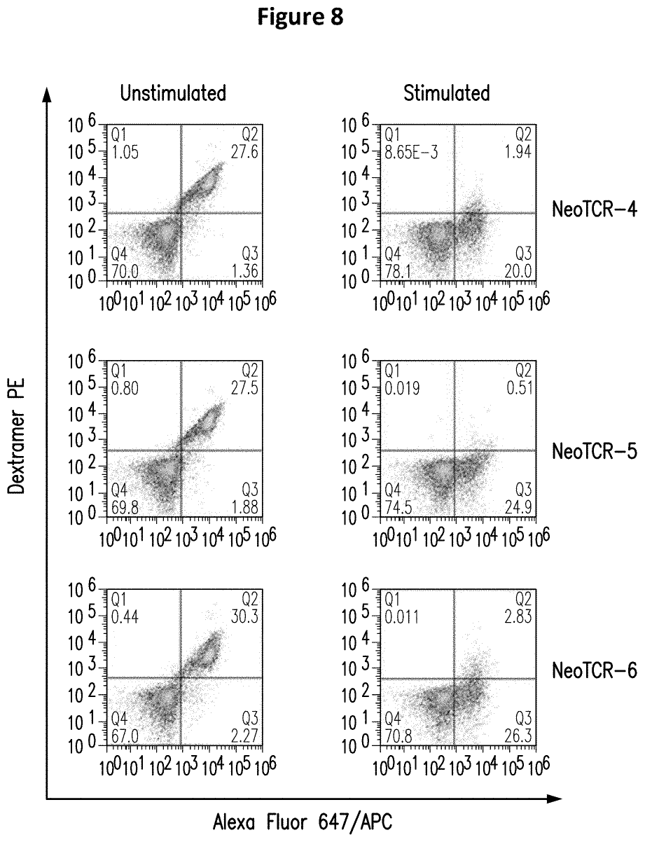

Another key reason why detection of the cleaved 2A peptide is important is that the TCR can be down-regulated during T cell stimulation and the NeoTCR edited cells cannot be identified through dextramer binding. The ability to detect the occurrence of gene editing using an anti-2A peptide antibody was also tested in unstimulated CD8 T cells and in stimulated CD8 T cells (FIG. 8). The stimulated CD8 T cells were stimulated with TransACT for 18 hours. The FACS analysis shown in FIG. 8 was gated as lymphocytes, singlets, live cells, and CD8+ cells and was performed on gene edited cells NeoTCR-4, NeoTCR-5, and NeoTCR-6. This shows that the NeoTCR is internalized upon stimulation (loss of staining in the upper right quadrant (Q2)) and thus showing minimal dextramer staining. Notwithstanding the foregoing, the anti-2A antibody was still able to detect the intracellular cleaved 2A peptide proving that an anti-2A peptide antibody can detect cleaved 2A peptide using FACS as a measure of detected gene edited cells. While TransAct was used for the experiments shown in FIG. 8, other activation mechanisms known to one of skill in the art can also be used, including but not limited to stimulation with CD3 and CD28 or with a cognate comPACT molecule.

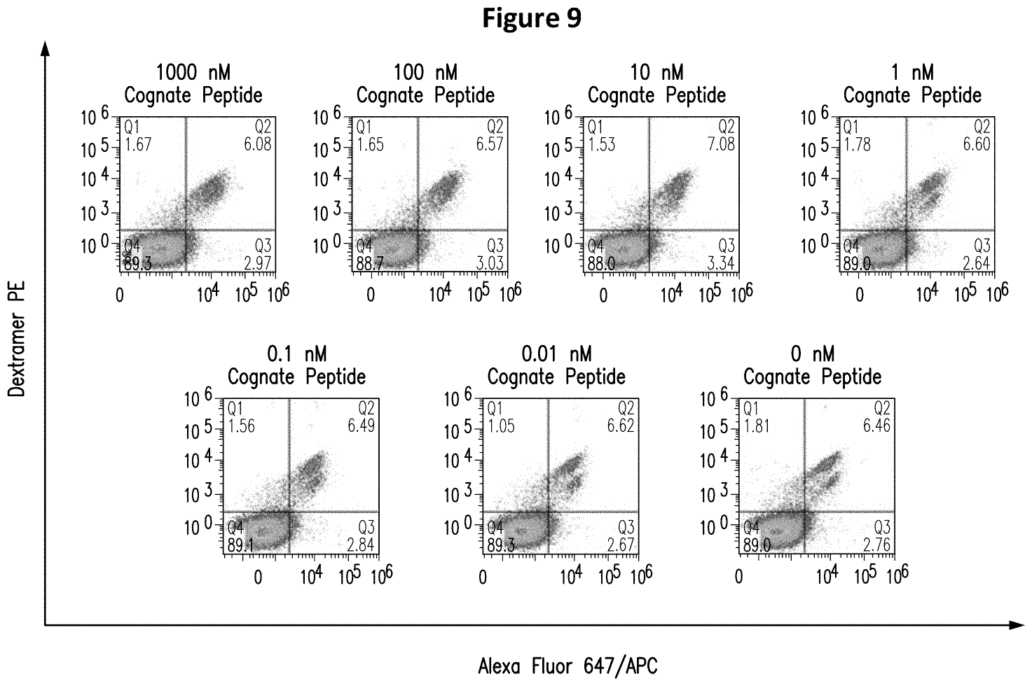

It was also confirmed that different levels of stimulation (using cognate peptide to the NeoTCR) of the gene edited T cells did not affect the ability to detect the cleaved 2A peptide by FACS using an anti-2A peptide antibody (FIG. 9). As shown, regardless of the amount of cognate peptide used to stimulate the gene edited T cells, the cleaved 2A peptide was detectable and unchanged based on the stimulation. Specifically, it was shown that stimulation appears to diminish intensity of cleaved 2A peptide detection using an anti-2A peptide antibody but the cleaved 2A peptide is still detectable on a similar (albeit slightly lower) percentage of cells, even when dextramer staining is essentially undetectable because of internalization.

In summary, 2A peptide staining with an anti-2A peptide antibody directly measures gene editing in cells, by binding to the 2A epitope tagged truncated NeoTCR alpha chain variable region polypeptide in the endoplasmic reticulum. Permeabilization of the cells is required for the antibody to access its epitope. This staining is specific, as it is not observed in mock transfected cells, and in gene edited cell populations for cells where both CD4 and CD8 T cells bind dextramer is only observed on the dextramer positive cells. This technique is effective for staining CD4 T cells to assess gene editing for NeoTCRs where dextramer does not bind to CD4 T cells. It is also effective to stain recently stimulated cells that have internalized their NeoTCRs to the point that they do not stain with dextramer. In this latter case, it is worth noting that the percentage of 2A+ cells is slightly reduced, as is the mean fluorescence intensity of staining. Overall, these experiments indicate that an anti-2A antibody can readily assess gene edited T cells directly using FACS.

### Example 5. Method of Detecting the Rate of Gene Editing (i.e., Percent of Cells Gene Edited or Ratio of Cells Gene Edited) Using an Anti-2A Antibody

Not only is it necessary to detect whether or not cells have been successfully gene edited, it is also necessary to be able to detect the rate of gene editing. This was done using the methods described in Example 4. Specifically, FACS analysis on a batch of cells that went through a gene editing process as disclosed in Examples 1-3, were stained with an anti-2A peptide antibody and the percent gene edited cells were calculated using FACS such that cells that stained with the anti-2A peptide antibody were determined to be gene edited and the cells that did not stain with the anti-2A peptide antibody were determined to be not edited. This allowed for determination of the efficiency of the gene editing process and a percent successful gene editing was determined using the following calculation: (number of cells positive for anti-2A antibody staining)÷(total number of cells).

Furthermore, the rate of gene editing was also be calculated using the formula described above and including NeoTCR staining (using dextramer) in addition to 2A staining (using an anti-2A peptide antibody). In such experiments, the NeoTCR staining is not included in the calculation of rate of gene editing.

Determining the rate of gene editing was used to optimize gene editing processes including but not limited to optimizing electroporation methods.

### Example 6. Method of Identifying and Tracking Edited Cells without NeoTCR Staining In Vitro and In Vivo

One critical step to drug development of cell therapy products that have undergone gene editing, is the step of tracking the existence and persistence of gene edited cells in culture (in vitro) and post-infusion into patients (in vivo).

For in vitro tracking the existence and persistence of gene edited cells, once the cells are gene edited using one or more 2A peptides (as described by way of example in Examples 1-3) and placed into culture, samples of the cultured cells were taken at various time point to determine whether the gene edited cells were expanding, surviving, and/or dying using FACS to detect the presence of the cleaved 2A peptide as described in Example 5. Based on the time points collected, culture conditions were adjusted in order to promote expansion and survival.

For in vivo tracking the existence and persistence of gene edited cells that included one or more 2A peptides in the gene editing process, it was possible to take blood samples from patients who had previously been infused with a drug product comprising the gene edited cells and detect the presence and persistence of the gene edited cells at varying time points post-infusion using FACS to detect the cleaved 2A peptide as described in Example 5.

In specific examples, cells were edited using the methods described in Examples 1-3 and then prepared as a drug product (a Cell Product) for the treatment of cancers in patients in need thereof. After the infusion, there was a need to track the edited cells to determine they engrafted and persisted in the patients or if they would die and be eliminated. This is critical because there was a need to know if the edited cells could persist and continue treating the patient by decreasing the tumor burden of the patient, eliminating the tumor burden of the patient, or preventing the continued expansion of the tumor burden of the patient (i.e., stable disease). By using the FACS sorting of anti-2A peptide stained T cells acquired from the patients through blood draws after infusion of the gene edited cells, it was determined that gene edited cells were present and detected.

### Example 7. Methods of Staining Gene Edited Cells with an Anti-2A Peptide Antibody and Dextramer

It was determined that the order of fixing, permeabilization, and staining affects the quality of analysis of gene edited cells based on anti-2A peptide antibody and dextramer staining. A number of staining methods were explored in order to optimize the ability to stain and detect dextramer positive and anti-2A peptide positive cells. Three examples of the various staining methods are provided in Table 2. In addition to the examples shown in Table 2, different iterations and variations of the staining methods were tested. In addition, experiments were performed that showed that cell permeabilization was needed for 2A peptide staining. Furthermore, it is necessary to perform the dextramer staining before permeabilization in order to properly detect cell surface expression of the NeoTCR.

TABLE 2
Three exemplary staining methods

| | | Method 2 | |
|---|---|---|---|
| | Method 1 | Fixation after | |
| | Fixation after | additional cell | Method 3 |
| Staining | dextramer | surface marker | Fixation for |
| Step | staining | staining | storage only |
| 1 | 20 min live/dead | 20 min live/dead | 20 min live/dead |
| | stain, wash | stain, wash | stain, wash |
| 2 | 10 min dextramer, | 10 min dextramer, | 10 min dextramer, |
| | wash | no wash | no wash |
| 3 | 5 min fix, wash | 20 min additional | 20 min additional |
| | | cell surface marker | cell surface marker |
| | | stains, wash | stains, wash |
| 4 | 20 min additional | 5 min fix, wash | 2A stain in |
| | cell surface marker | | permeabilization |
| | stains, wash | | buffer |
| 5 | 2A peptide stain | 2A peptide stain | fix for storage |
| | in permeabilization | in permeabilization | |
| | buffer | buffer | |
| 6 | fix for storage | fix for storage | N/A |

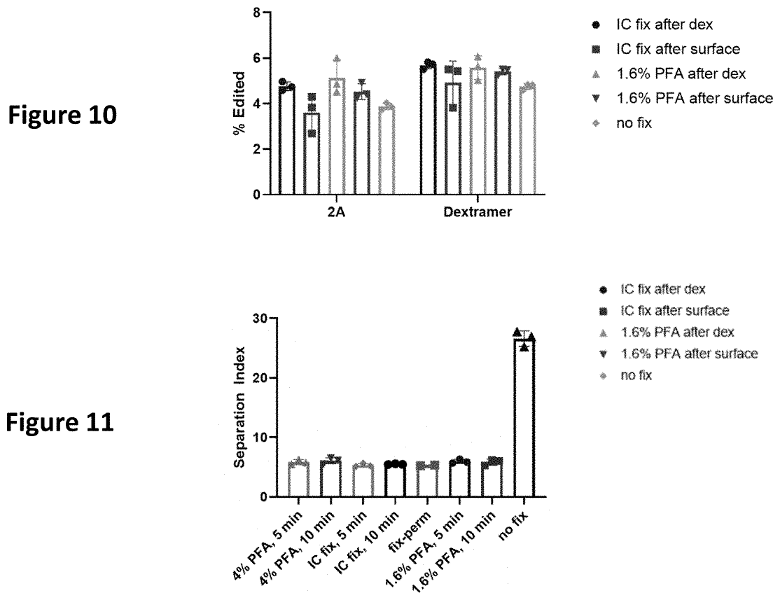

As shown in Table 2, different orders of three exemplary staining conditions. The first step for each Method includes a 20 min live/dead stain which includes staining the cells with live/dead viability dye followed by washing to remove the excess dye. In Methods 1 and 2, the order of the mid-process fixation is different: Method 1 providing a fixation step in between the dextramer stain and the additional cell surface marker stains while Method 2 provides for the fixation step after the additional cell surface marker stains. A key difference between Methods 1 and 2 and that of Method 3 is that there is no mid-process fixation step in Method 3. In other words, Methods 1 and 2 provide a fixation step prior to the 2A peptide stain and Method 3 does not have a fixation step prior to the 2A peptide stain. Results from these experiments are shown in FIG. 10. As shown, two different fixation methods were used in this experiment: 1) IC Fixation Buffer (“IC fix”) and 2) 1.6% Paraformaldehyde (“PFA”). The IC fix was designed as an intracellular fixation buffer for use in flow cytometry, particularly for fixation in intracellular staining procedures and was composed of paraformaldehyde in phosphate buffered saline, pH 7.3 (ThermoFisher Cat No. FB001). As shown in FIG. 10, all conditions resulted in the ability to detect both the cleaved 2A peptide (anti-2A peptide antibody) and the NeoTCR (dextramer).

However, the separation index between 2A peptide detection and NeoTCR detection shows that use of fixation prior to the 2A peptide staining with an anti-2A antibody significantly lowers the separation index compared to a process without the mid-process fixation step as described above (FIG. 11). The decreased separation index between the dextramer (TCR) detection and the 2A peptide detection (anti-2A peptide antibody) using FACS is the result of poorer 2A peptide staining and detection. This is likely because the fixation modifies the 2A peptide epitope. Nonetheless, the cleaved 2A peptide was still detectable and it was still possible to perform experiments as described in Example 3 wherein the question of whether a cell that did not have dextramer (TCR) signal detected using FACS was in fact gene edited but the NeoTCR was a low affinity binder or if the cell was not gene edited.

It was also confirmed that when the mid-process fixation step was removed, detection of other surface antibodies (such as TCR) could be impacted and there appeared to be a loss of dextramer binding. Accordingly, it is necessary to include or exclude the mid-process fixation step based on the question that needs answering. For experiments that require high quality staining of surface markers, the mid-process fixation step can be selected and used. In fact, such experiments were performed and confirmed. For experiments that require the highest quality 2A peptide detection (for example, if the question of what percent of cells were edited as discussed in Examples 5 and 6), the process without a mid-process fixation step can be selected and used. In fact, such experiments were performed and confirmed. In experiments where less high-quality detection, but sufficient detection nonetheless of both the 2A peptide and the NeoTCR (e.g., for qualitative experiments), either a process with a mid-process fixation step or a process without a mid-process fixation step can be used. In fact, such experiments were performed and confirmed. Based on experiments performed using multiple conditions, it was determined that performing the FACS assay using the process described in Method 3 of Table 2 was a preferred method to use when the goals are to 1) determine if the cells were edited using 2A detection, 2) qualitatively confirm that the NeoTCR is present on the cells in support of the gene editing detection (the 2A detection) using dextramer, and optionally 3) when additional functional assays are performed to confirm the activity of the NeoTCR that was gene edited into the cells.

The final approach which was employed with performing separate assays: a first FACS assay for the cleaved 2A peptide detection and a second FACS assay for the NeoTCR. In this scenario, the cells were split for two separate assays. This provides the ability to achieve high-quality quantitative data for both the cleaved 2A peptide and the NeoTCR.

### Example 8: Method of Detecting and Tracking Cell Products In Vivo & In Vitro

Any of the methods described in Examples 5-7 can be used for any Cell Product. While NeoTCR Products were used for exemplary purposes, methods of detecting and tracking gene edited cells in vivo and in vitro, the in vivo and in vitro methods described in Examples 5-7 can be used for any Cell Product.

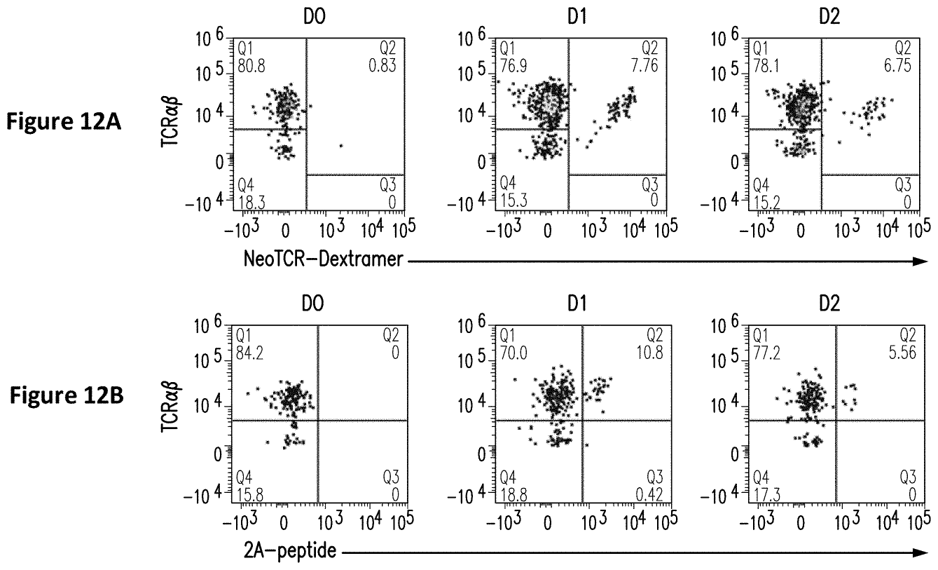

Furthermore, the methods described in Examples 5-7 were used to detect presence and persistence of the Cell Product in a patient to determine if the cells of the Cell Product were remaining in the patient as persistent cells. As shown in FIGS. 12A and 12B, NeoTCR+ T cells were detected in blood samples from patients who were administered a NeoTCR Product on days 1-2 after infusion of a NeoTCR Product using dextramer staining and 2A peptide staining. This confirms that the NeoTCR Products persisted in patients post infusion for 2 days post infusion by detecting the NeoTCR itself and by the 2A peptide which confirms that the NeoTCR was not an endogenous TCR and rather the gene edited product.

Furthermore, the methods described in Examples 5-7 were used to calculate the pharmacokinetic (PK) profile of Cell Products in patients post-infusion. Specifically, the PK profile of NeoTCR Products was determined from patients infused with the NeoTCR Products (including but not limited to half-life, Cmax, volume distribution, Tmax, Cmin, concentration, elimination half-life, elimination rate constant, clearance, and area under the curve).

Furthermore, the methods described in Examples 5-7 can be used to detect expansion of the Cell Product in a patient to determine if the cells of the Cell Product are dividing and expanding in vivo, in the patient.

Furthermore, the methods described in Examples 5-7 have been used to detect expansion of the Cell Product in a culture condition in vitro to determine if the cells of the Cell Product were dividing and expanding in vitro.

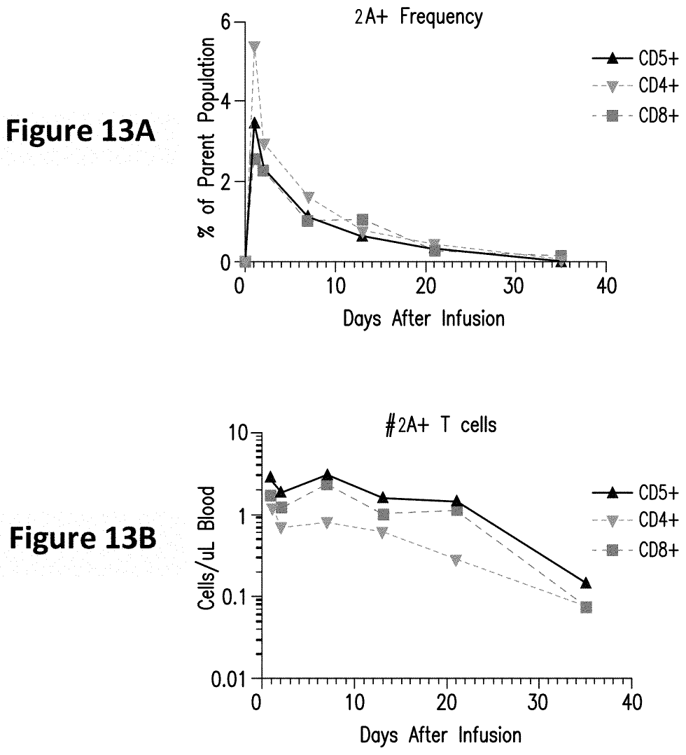

As shown in FIGS. 13A and 13B (data from the same patient), the 2A Diagnostics described herein used flow cytometry to detect gene-edited cells in the blood of a human patient post-infusion. Specifically, detection of gene-edited cells was performed using the flow cytometry methods described herein with an antibody that detects the sequence of SEQ ID NO:1. FIG. 13A shows the frequency of 2A positive T cells (CD4+, CD8+ and CDS+) was tracked and detected for 35 days in an experiment performed on one patient that treated with and administered a NeoTCR Product. FIG. 13B shows the number of 2A positive T cells (CD4+, CD8+ and CD5+) per microliter of blood over a period of 35 days following the administration of a NeoTCR Product to a patient.

In addition flow cytometry based detection, a 2A Diagnostic was performed on tissue using IHC. For the IHC 2A Diagnostic experiments, the following methods were use: 1) cell pellets were made (20M or 30M cells/vial) and were composed of gene edited (2A positive) and not gene edited CD4 and CD8 T cells as indicated in Table 3; 2) the pellets were cryopreserved in cryopreservation media formulation (a mix 1:1 (v/v) of Cryostor CS10: Plasma-Lyte A+2% Human Serum Albumin (w/v)); 3) the cell pellets were thawed and placed in a centrifuge tube and spun to remove excess fluid; 4) the cells were aliquoted, washed with PBS, and resuspended in 10% NBF (neutral buffered formalin) overnight at 4° C.; 5) the cells were washed with PBS and pelleted; 6) 150 μL of pre-warmed Histogel (or alternative standard histological reagent) (65° C.) was added to the cell pellet and vortexed immediately to disperse the cells; 7) 70% EtOH was added to the tube; 8) the cells were embedded in paraffin; 9) the FFPE blocks of embedded cells were sectioned at 5 μm thickness onto FFPE coverslips; 10) sections on coverslips were deparaffinized and rehydrated using Citrisolv solution followed by a decreasing series of concentrated ethanol; 11) sections then underwent antigen retrieval in a tris-based buffer with a slightly basic pH (CCl solution from Ventana), followed by heat-induced epitope retrieval (HIER) in a TintoRetriever pressure cooker at 95° C.; and 12) coverslips were then loaded onto ZellSafe Chips and subsequently stained for ChipCytometry analysis.

TABLE 3
IHC Cell Pellet Compositions

| | Total | | | | |
|---|---|---|---|---|---|
| | 2A % | % CD4 | % CD8 | CD4 + 2A+ | CD8 + 2A+ |
| Neo12 TCR | 58 | 20 | 78 | 43 | 62 |
| TCR 1 | 57 | 25 | 73 | 51 | 60 |
| TCR 2 | 59 | 19 | 79 | 60 | 60 |
| TCR 3 | 57 | 23 | 74 | 51 | 60 |

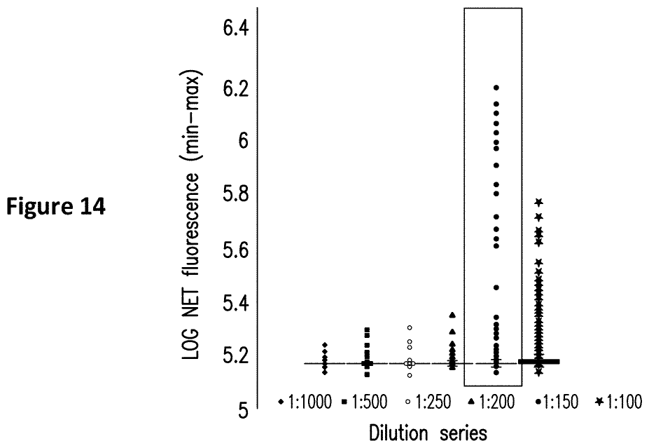

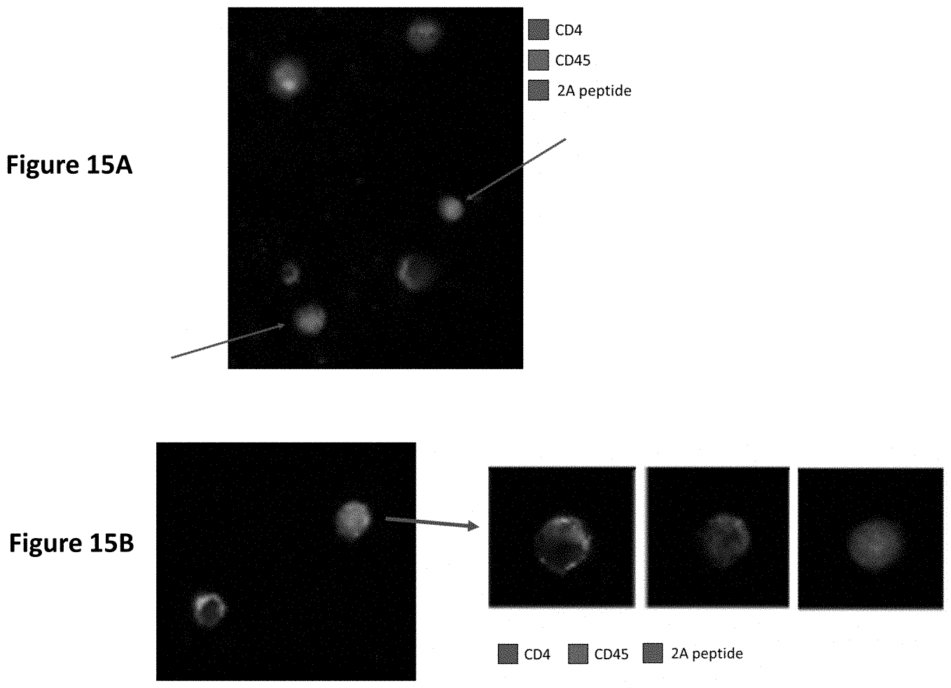

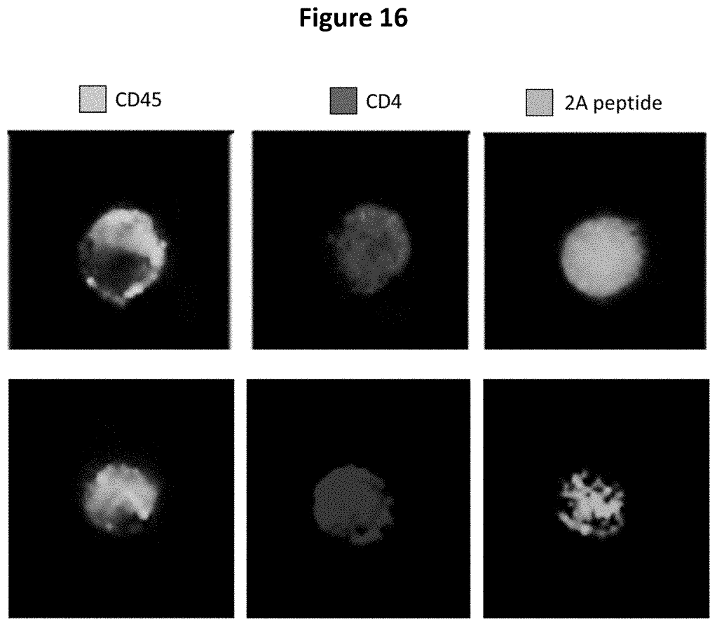

As shown in FIG. 14, the 3H4 anti-2A antibody (FITC-conjugated) was tested at serial dilutions to find the optimal staining ratio. It was determined that for the 3H4 FITC-conjugated antibody, the 1:150 dilution was optimal but that all other dilutions (preferably 1:200-1:100) were also suitable for certain 2A Diagnostics. Using the 1:150 dilution, CipCytometry was performed on slides made from cells expressing the control Neo12 TCR and TCR 1, TCR 2, and TCR 3 from patient samples. Representative images of the 2A staining in sections of FFPE cell pellets are shown in FIGS. 15A and 15B (FIG. 15B showing a magnified image of a cell from FIG. 15A). These images show that the 2A Diagnostic was successful at detecting gene edited cells (using an anti-2A antibody) with an IHC assay. Furthermore, FIG. 16 provides two different images of the same cell (the bottom row showing a cross section view of the cell) which shows the successful detection of intracellular 2A peptide (specifically, the cleaved fragment of the 2A peptide).

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

1. A method of determining a presence of an adoptive cell therapy in a subject, comprising: detecting an expression level of a 2A peptide in a sample from the subject that has received the adoptive cell therapy, wherein the adoptive cell therapy comprises a cell expressing the 2A peptide, or a fragment thereof.

2. The method of claim 1, wherein
a) detection of the 2A peptide, or a fragment thereof, is associated with the presence of the adoptive cell therapy; or
b) absence of the 2A peptide, or a fragment thereof, is associated with inefficiency of the adoptive cell therapy.

3. (canceled).

4. The method of claim 1, wherein detecting of the 2A peptide, or a fragment thereof, occurs between 1 day and 30 days, between 1 month and 12 months, or between 1 year and 5 years after the subject has received the adoptive cell therapy.

5.-38. (canceled).

39. A method of treating a cancer in a subject that has received an adoptive cell therapy, wherein the adoptive cell therapy comprises a cell expressing a 2A peptide, or fragment thereof, the method comprising:
a) detecting the expression level of the 2A peptide, or fragment thereof, in a sample from the subject, wherein absence or decreased detection of the 2A peptide, or fragment thereof, is associated with inefficiency of the adoptive cell therapy; and
b) administering a second adoptive cell therapy, when the expression level of the 2A peptide is absent or decreased.

40. The method of claim 39, wherein detecting of the 2A peptide, or fragment thereof, occurs between 1 day and 30 days, between 1 month and 12 months, or between 1 year and 5 years after the subject has received the adoptive cell therapy.

41.-43. (canceled).

44. The method of claim 39, wherein the cell is a NeoTCR Product.

45. The method of claim 39, wherein the sample is a blood sample or a tumor sample.

46. (canceled).

47. The method of claim 39, wherein the subject is human.

48.-50. (canceled).

51. The method of claim 39, wherein the 2A peptide, or fragment thereof, is a cleaved 2A peptide.

52. The method of claim 39, wherein the 2A peptide is selected from the group comprising P2A, T2A, E2A, and F2A peptides.

53.-54. (canceled).

55. The method of claim 39, wherein the 2A peptide, or fragment thereof, is detected using an anti-2A antibody or antigen binding portion thereof.

56. The method of claim 55, wherein the anti-2A antibody cross-competes for binding to the 2A peptide with a reference antibody or antigen-binding portion thereof, wherein the reference antibody or antigen-binding portion is a 3H4 antibody.

57. The method of claim 55, wherein the anti-2A antibody binds to the same epitope of the 2A peptide with a reference antibody or antigen-binding portion thereof, wherein the reference antibody or antigen-binding portion thereof, wherein the reference antibody or antigen-binding portion is a 3H4 antibody.

58. The method of claim 55, wherein the anti-2A antibody cross-competes for binding to the 2A peptide with a reference antibody or antigen-binding portion thereof, wherein the reference antibody or antigen-binding portion is an ABS31 antibody.

59. The method of claim 55, wherein the anti-2A antibody binds to the same epitope of the 2A peptide with a reference antibody or antigen-binding portion thereof, wherein the reference antibody or antigen-binding portion thereof, wherein the reference antibody or antigen-binding portion is an ABS31 antibody.

60. The method of claim 55, wherein the anti-2A antibody or antigen-binding portion thereof is a 3H4 antibody.

61. The method of claim 55, wherein the anti-2A antibody or antigen-binding portion thereof is an ABS31 antibody.

62. The method of claim 55, wherein the anti-2A antibody binds to an epitope having an amino acid sequence set forth in SEQ ID NO: 3.

63.-66. (canceled).

67. A method of lot releasing a population of cells for adoptive cell therapy, comprising: transducing or transfecting the population of cells with a vector comprising an antigen binding receptor and a 2A peptide; detecting an amount of cells expressing the 2A peptide, or fragment thereof; releasing the population of cells for therapeutic use when the amount of cells expressing the 2A peptide is above a threshold.

68. (canceled).

69. The method of claim 67, wherein the vector comprises:
a) first and second homology arms homologous to first and second target nucleic acid sequences and oriented to facilitate homologous recombination;
b) a nucleotide sequence encoding the TCR positioned between the first and second homology arms; and
c) a first nucleotide sequence encoding a 2A ribosome skipping element positioned upstream of the nucleotide sequence encoding the TCR polypeptide and a second nucleotide sequence encoding a 2A ribosome skipping element positioned downstream of the nucleotide sequence encoding the TCR polypeptide, wherein the first and second nucleotide sequences encoding the 2A ribosome skipping elements have the same amino acid sequences and are codon-diverged relative to each other.

70.-114. (canceled).
